(19) United States
(12) Patent Application Publication
(10) Pub. No.: US 2016/0015750 A1
(43) Pub. Date: Jan. 21, 2016

(54) VASCULAR-TARGETED T-CELL THERAPY

(71) Applicant: BAYLOR COLLEGE OF MEDICINE, Houston, TX (US)

(72) Inventors: Stephen M. G. Gottschalk, Houston, TX (US); LaTerrica C. Williams, Houston, TX (US); Tiara T. Byrd, Houston, TX (US); Nabil M. Ahmed, Houston, TX (US); Xiao-Tong Song, Pearland, TX (US)

(21) Appl. No.: 14/773,434

(22) PCT Filed: Mar. 10, 2014
(86) PCT No.: PCT/US2014/022751

§ 371 Date: Sep. 8, 2015

Related U.S. Application Data
(60) Provisional application No. 61/775,541, filed on Mar. 9, 2013.

(51) Int. Cl. A61K 35/17; C07K 14/725; C07K 14/705

(52) U.S. Cl. CPC A61K 35/17; C07K 14/70521; C07K 14/7051; C07K 14/70578; A61K 2035/124

(57) ABSTRACT

Embodiments of the invention provide for cell therapy for cancers having a TEM1 or TEM8 antigen. Certain embodiments provide for cell therapy that targets tumor vasculature, including the tumor vascular bed, for example. In specific embodiments, TEM1- and/or TEM8-specific chimeric antigen receptors are employed.

This application claims priority to U.S. Provisional Patent Application Ser. No. 61/775,541, filed Mar. 9, 2013, which is incorporated by reference herein in its entirety.

### STATEMENT REGARDING FEDERALLY SPONSORED RESEARCH OR DEVELOPMENT

This invention was made with government support under R01 CA148748, 5T32HL092332, and P01 CA094237 awarded by NIH. The government has certain rights in the invention.

### TECHNICAL FIELD

Embodiments of the invention concern at least the fields of immunology, cell biology, molecular biology, and medicine, including cancer medicine.

### BACKGROUND OF THE INVENTION

Immunotherapy with antigen-specific T cells has shown promise in the treatment of malignancies in preclinical models as well as in Phase I/II clinical studies. One attractive strategy to generate tumor-specific T cells is by genetic modification with chimeric antigen receptors (CARs), which comprise an extracellular antigen recognition domain, a transmembrane domain, and an intracellular signaling domain derived from the T-cell receptor CD3-zeta chain often linked to costimulatory molecule endodomains.

The majority of CAR-T cells approaches have focused on targeting antigens expressed on tumor cells. However, targeting other components of tumors is critical for complete eradication and to prevent recurrences. Targeting the vasculature bed, one component of the stroma, has shown promise in preclinical as well as clinical studies leading to the FDA approval of bevacizumab, an antibody against vascular endothelial growth factor, for the treatment of several solid tumors and brain tumors. However, most patients treated with bevacizumab or other angiogenesis inhibitors ultimately develop recurrent and/or progressive disease. This lack of efficacy is most likely explained by the redundancy of angiogenesis pathways. In addition, recent studies indicate that part of the tumor vasculature can be derived from the malignant cells themselves, necessitating a more aggressive ‘angiotoxic approach’ to eradicate the vasculature in GBMs. “Angiotoxic” therapies include the use of immunotoxins or genetically engineered T cells. These are considered more potent than anti-angiogenesis agents because they kill endothelial cells, resulting in vessel thrombosis.

The present invention provides a novel solution in the art of delivering effective cancer therapies to individuals in need thereof.

### BRIEF SUMMARY OF THE INVENTION

The present invention is directed to methods and compositions related to cell therapy. In particular embodiments, the cell therapy is for an individual in need of cell therapy, such as a mammal, including a human.

In a first aspect, provided herein are genetically engineered immune cells, e.g., T cells (T lymphocytes), natural killer (NK) cells or NK T cells, that are directed to a tumor vasculature antigen, e.g., tumor endothelial marker 1 (TEM1) or TEM8. TEM1 is also referred to in the art as endosialin or CD248. In a specific embodiment, the genetically engineered immune cells are T cells. In specific embodiments, the genetically engineered immune cells, e.g., T cells, comprise a receptor that (1) targets the immune cell to tumor vasculature and (2) stimulates the immune cell to kill tumor vasculature cells. In more specific embodiments, the receptor is a chimeric antigen receptor (CAR) that directs the immune cells to the tumor vasculature antigen, e.g., TEM1 or TEM8, on tumor vasculature cells. In specific embodiments, when the CAR binds to the tumor vasculature antigen, the immune cell kills the tumor vasculature cells. In certain embodiments, the immune cell, e.g., T cell, comprises a CAR that binds to TEM1, or a CAR that binds to TEM8, or a CAR that binds to TEM1 and a CAR that binds to TEM8.

In certain embodiments, a CAR that directs an immune cell to TEM1 or TEM8 comprises (1) an extracellular antigen-binding domain that binds to TEM1 or TEM8, and (2) an intracellular domain that comprises a primary signaling moiety, e.g., a CD3ζ chain, that provides a primary T cell activation signal, and optionally a costimulatory moiety, e.g., a CD28 polypeptide and/or a 4-1BB (CD137) polypeptide. In certain specific embodiments, the T cell comprises a first CAR that provides a primary T cell activation signal, and a second CAR that provides a costimulatory signal. In various specific embodiments, the first CAR binds to TEM1 and provides a primary T cell activation signal, and the second CAR binds to TEM8 and provides a costimulatory signal; the first CAR binds to TEM8 and provides a primary T cell activation signal, and the second CAR binds TEM1 and provides a costimulatory signal; the first CAR binds TEM1 and provides a primary T cell activation signal and the second CAR binds a tumor associated antigen (TAA) or tumor specific antigen (TSA) and provides a costimulatory signal; the first CAR binds a TAA or TSA and provides a primary T cell activation signal and the second CAR binds TEM1 and provides a costimulatory signal; the first CAR binds TEM8 and provides a primary T cell activation signal and the second CAR binds a TAA or TSA and provides a costimulatory signal; the first CAR binds a TAA or TSA and provides a primary T cell activation signal and the second CAR binds TEM8 and provides a costimulatory signal.

In another aspect, provided herein are any of the TEM1 and/or TEM8 CAR polypeptides described herein. Also provided are polynucleotides encoding such CARs. In embodiments of the invention, there is a polynucleotide comprising sequence that encodes a TEM1-specific chimeric antigen receptor (CAR).

In embodiments of the invention, there is a polynucleotide comprising sequence that encodes a TEM8-specific CAR.

Any polynucleotide of the invention may comprise sequence that encodes a TEM8-specific CAR. Any CAR may comprise a transmembrane domain selected from the group consisting of transmembrane proteins including but not limited to CD3-zeta or CD28. A CAR may comprise one or more co-stimulatory molecule endodomains selected from the group consisting of costimulatory molecules including but not limited to CD28, CD27, 4-1BB, OX40 ICOS, and a combination thereof. Any polynucleotide of the invention may be comprised in an expression vector, including one that is a viral vector, such as a retroviral vector, lentiviral vector, adenoviral vector, or adeno-associated viral vector. In embodiments of the invention, there is a cell, comprising at least one of any expression vector of the invention. The cell may be a eukaryotic or prokaryotic cell. The cell may be an immune system cell. The cell may be a T cell, NK cell, or NKT cell.

In another aspect, provided herein are methods of treating an individual having a disease or disorder associated with, or caused by, pathological angiogenesis. In certain embodiments, the disease or disorder is cancer, e.g., solid tumors. In certain other embodiments, the disease or disorder is one or more of an inflammatory disorder, arthritis, psoriasis, endometriosis, atherosclerosis, hemangiomas, or other hamartomatous conditions.

In embodiments in which the disease or disorder is cancer, the cancer may be of any kind and of any stage. The individual having cancer may be of any age or either gender. In specific embodiments, the individual is known to have cancer, is at risk for having cancer, or is suspected of having cancer. The cancer may be a primary or metastatic cancer, and the cancer may be refractory to treatment with other modalities, e.g., chemotherapy, radiation, or the like. In specific embodiments, the cancer is leukemia, lymphoma, myeloma, breast cancer, lung cancer, brain cancer (e.g., a glioma or medulloma), colon cancer, kidney cancer, prostate cancer, pancreatic cancer, thyroid cancer, bone cancer, cervical cancer, cancer of the spleen, anal cancer, esophageal cancer, head and neck cancer, stomach cancer (gastric cancer), gall bladder cancer, melanoma, non-small cell lung cancer, and so forth, for example. In particular aspects, the cancer expresses one or more tumor antigens, e.g., TAA or TSA, although upon identification of a type of cancer in an individual, the presence of the particular tumor antigen(s) may or may not be verified.

In certain embodiments of the invention, the invention concerns methods and compositions related to therapeutic cells, including therapeutic immune system cells such as tumor-specific cytotoxic T lymphocytes. The cells may be T-cells, NK cells or NKT cells, as well as other cellular elements with the capability of inducing an effector immune response. In certain aspects, the cells express at least one non-endogenous molecule that targets a particular tumor antigen, and in at least some cases, the molecule comprises a single chain variable fragment (scFv). In particular aspects, the molecule is a receptor. The receptor is a chimeric antigen receptor (CAR), in particular embodiments. In specific aspects, the CAR is directed to at least one antigen on the tumor vasculature. Although any tumor vasculature antigen may be targeted in the invention, in specific embodiments the tumor vasculature antigen is Tumor endothelial marker (TEM)1 or TEM8 (or both). Other tumor vasculature antigens may be targeted concomitantly, such as with other immunotherapy (including antibodies or other types of CARs) or chemotherapy, for example.

In embodiments of the invention, there is a method of treating an individual for cancer, comprising the step of providing a therapeutically effective amount of a plurality of any of cells of the invention. The cancer may comprise solid tumors, including solid tumors that are about 2 mm or greater in diameter. The cancer may be lung, bronchial, breast, prostate, intestine (including esophagus, stomach, small intestine, colon, rectal, and anal), brain and nervous system, eye (including retinoblastoma), neuroectodermal, skin, liver including bile and gallbladder, kidney, bladder, pancreatic, blood, thyroid, gynecological including cervical and ovarian, testicular, stomach, spleen, gall bladder, soft tissue (sarcoma), bone, endocrine, undifferentiated, oral cavity, head and neck, oral cavity, primary and secondary nervous system malignancies of various histologies, viral (including AIDS, EBV, HPV)-related, or undifferentiated. In any method of the invention, it may further comprise the step of providing a therapeutically effective amount of an additional cancer therapy to the individual.

Embodiments of the invention include methods to target the tumor vascular bed, in addition to tumor cells, with particular CART-cells. Such methods enhance the in vivo antitumor activity of therapies for a broad range of malignancies, in at least certain cases. Examples of targetable antigens in the tumor bed, but not limited to, include tumor endothelial markers (TEMs), vascular endothelial growth factor receptors (VEGFRs), endoglin, and integrins

Cells of the invention that may be modified to target tumor vasculature include at least T-cells (which may be referred to as cytotoxic T lymphocytes (CTLs)), NK-cells, NKT-cells, or any other cellular elements with the capability of inducing an effector immune response. In particular cases the cells harbor a polynucleotide that encodes the CAR.

In embodiments of the invention there is a kit comprising at least one polynucleotide of the invention, at least one expression vector of the invention, and/or at least one cell or cells of the invention.

In one embodiment, there is a polynucleotide comprising sequence that encodes a TEM1-specific chimeric antigen receptor. In another embodiment, there is a polynucleotide comprising sequence that encodes a TEM8-specific chimeric antigen receptor. A polynucleotide of the disclosure may further comprise sequence that encodes a TEM8-specific chimeric antigen receptor. In specific embodiments, the chimeric antigen receptor comprises a transmembrane domain selected from the group consisting of CD3-zeta and CD28. In certain embodiments, the chimeric antigen receptor comprises co-stimulatory molecule endodomains selected from the group consisting of CD28, CD27, 4-1BB, OX40 ICOS, and a combination thereof. In embodiments of the disclosure, there is an expression vector comprising a polynucleotide of the disclosure. In specific embodiments, the vector is a viral vector, such as a retroviral vector, lentiviral vector, adenoviral vector, or adeno-associated viral vector. In some embodiments, there is a cell, comprising a expression vector of the disclosure. The cell may be a a eukaryotic cell, such as an immune system cell, including a T cell, NK cell, or NKT cell.

In certain embodiments, there is a method of treating an individual for cancer, comprising the step of providing a therapeutically effective amount of a plurality of any of cells of the disclosure. In specific embodiments, the cancer is a solid tumor, such as a solid tumors that are about 2 mm or greater in diameter. In a specific embodiments, the cancer is lung, bronchial, breast, prostate, intestine (including esophagus, stomach, small intestine, colon, rectal, and anal), brain and nervous system, eye (including retinoblastoma), neuroectodermal, skin, liver including bile and gallbladder, kidney, bladder, pancreatic, blood, thyroid, gynecological including cervical and ovarian, testicular, stomach, spleen, gall bladder, soft tissue (sarcoma), bone, endocrine, undifferentiated, oral cavity, head and neck, oral cavity, primary and secondary nervous system malignancies of various histologies, viral (including AIDS, EBV, HPV)-related, or undifferentiated. In some embodiments of methods of the disclosure, the methods further comprise the step of providing a therapeutically effective amount of an additional cancer therapy to the individual. Embodiments of the disclosure include kits that comprise a polynucleotide of the disclosure, an expression vector of the disclosure, and/or cells of the disclosure.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows an exemplary structure of the retroviral vector encoding the TEM8-CAR. LTR: long terminal repeat; CH2CH3: hinge; TM: transmembrane domain

FIG. 2 shows an exemplary method to generate T cells expressing CARs

FIG. 3A shows a representative Fluorescence-activated cell sorting (FACS) plot of non-transduced and tranduced cells, and FIG. 3B shows summary FACS data for 5 donors.

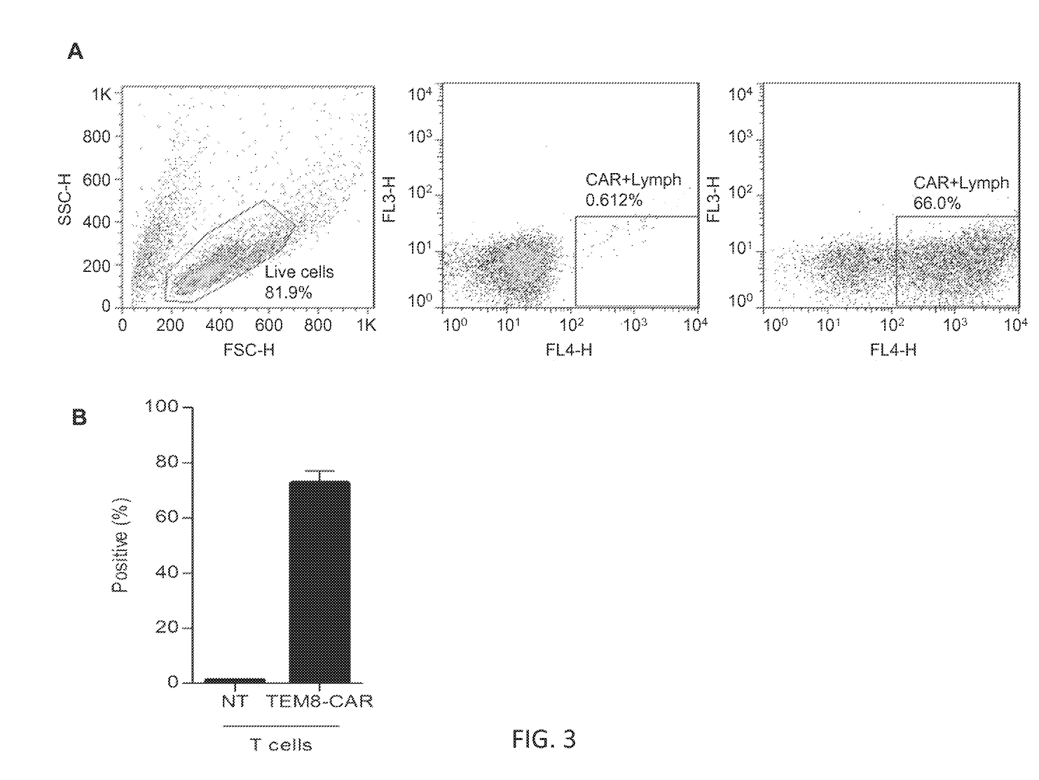

FIG. 4A show by Western blot that parental 293T cells do not express TEM8, while TEM8 expression is readily detectable in 293T cells that are genetically modified to express TEM8. FIG. 4B shows TEM8 expression in a panel of tumor cells by quantitative reverse transcriptase PCR (qRT-PCR).

FIG. 5A demonstrates that TEM8-CAR T cells produce IFN in the presence of TEM8-positive tumor cells. FIG. 12B demonstrates that TEM8-CAR T cells produce IL2 in the presence of TEM8-positive tumor cells.

FIG. 6 demonstrates that TEM8-CAR T cells kill TEM8-positive cells in a cytotoxicity assay.

FIG. 7 shows that TEM8-CAR T cells only kill TEM8-positive cells in a co-culture assay.

FIG. 8 shows an exemplary structure of the retroviral vector encoding the TEM8-CAR. LTR: long terminal repeat; CH2CH3: hinge; TM: transmembrane domain.

FIG. 9A demonstrates that TEM1-CAR T cells produce IFN in the presence of TEM1-positive tumor cells, and FIG. 9B demonstrates that TEM1-CAR T cells produce IL2 in the presence of TEM1-positive tumor cells.

FIG. 10 demonstrates that TEM8-CAR T cells kill TEM8-positive cells in a cytotoxicity assay.

FIG. 11 illustrates an example of a transgene to generate a TEM8 CAR and a corresponding plasmid.

FIG. 12 demonstrates with green fluorescence protein that the TEM8 CAR is expressed on the cell surface following introduction into the cell.

FIGS. 13A and 13B illustrate two methods for detection of a CAR on a cell surface.

FIG. 14 illustrates expression of TEM8 CARs on the surface of exemplary HEK 293T cells.

FIG. 15 demonstrates expression of TEM8 CARs on the surface of primary T cells.

FIG. 16 shows that TEM8 CAR T cells recognize and kill selected targets.

FIG. 17 shows that TEM8 CAR T cells selectively kill antigen-positive targets.

FIG. 18 demonstrates that TEM 8 CAR T cells recognize selected targets.

FIG. 19 demonstrates that TEM 8 CAR T cells recognize selected targets.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein embodiments which are disclosed and still obtain a like or similar result without departing from the spirit and scope of the invention.

### I. TEM1 and TEM8

The tumor microenvironment is quite complex, comprising different tumor elements such as cancer cells, cancer stem cells, endothelial cells, cancer-associated fibroblasts, pericytes, immune inflammatory cells, and/or invasive cancer cells, for example.

Solid tumors need blood vessels to grow beyond ˜2 mm. Once tumors reach an estimated size of ˜2-3 mm they must undergo an angiogenic switch. Angiogenesis is the formation of new blood vessels from existing blood vessels. After this switch has occurred, tumors are then able to produce their own blood vessels. Therefore, blood vessels are important for tumors to grow. Tumors secrete angiogenic factors (such as VEGF and/or angiopoietin) to form vasculature.

Although in normal vasculature the blood flow travels from the artery to arteriole to capillary bed to venule to veins in an organized fashion, tumor vasculature is very leaky and disorganized. This is because the tumor vasculature differs morphologically and functionally, and blood flow is sluggish and bidirectional. Tumor vessel networks are chaotic, disorganized, and contain abnormal irregular branches. In normal blood vessels, the tubes are non-permeable because the endothelial cells therein contain tight junctions. However, in tumor vasculature the tubes are leaky, dilated and highly permeable.

To be able to target tumor vasculature for therapeutic applications, a marker is needed that is specific and unique to the tumor vasculature, such as TEM1 and TEM8. In fact, TEMs are overexpressed in the neovasculature of many solid tumors, including colon, lung, and ovarian cancers, but expression in normal tissue is low.

TEM8 is associated with tumor progression, promoting tumor angiogenesis, and tumor growth but is dispensable for normal development and wound healing. It assists in endothelial tube formation and migration, and it is 96% homologous to murine TEM8, mTEM8. TEM8 is also called anthrax toxin receptor 1 and is highly specific to tumor endothelial cells. TEM8 is also part of the survival response activated by tumor microenvironment stress. It may function to block apoptosis in tumor endothelial cells that are growth factor-deprived or nutrient-deprived.

TEM1 (also known as endosialin/CD248) is a transmembrane protein of 757aa in length. The human protein has 77% homology to mTEM1. It plays a unique role in tumor progression and is involved in promoting angiogenesis, migration, proliferation and metastasis. TEM1 knockout mice have reduced tumor growth, reduced invasion and metastasis, yet retain normal wound healing. The gene is overexpressed at least in colon, breast, pancreatic and lung cancer. Expression in solid tumors has been identified, including colorectal carcinoma, lung adenocarcinoma, colon adenoCarcinoma, ovary carcinoma, and kidney clear-cell carcinoma, for example.

An exemplary TEM1 polynucleotide is provided in GenBank® Accession No. NM_{—}020404, and an exemplary TEM1 polypeptide is provided in GenBank® Accession No. NP_{—}065137, both of which are incorporated by reference herein in their entirety.

Tumor endothelial marker 8 (TEM8), also known as anthrax toxin receptor 1 (ANTXR1), is a highly conserved cell-surface protein overexpressed on tumor-infiltrating vasculature. TEM8 is an appealing target for selective inhibition of tumor angiogenesis because it is functionally required for optimal tumor angiogenesis and growth but unessential for normal development and physiological angiogenesis. Function-blocking antibodies specific to TEM8 (extracellular domain) inhibits pathological angiogenesis and tumor growth and supplements the activity of a variety of types of anticancer agents, including VEGFR inhibitors, for example. Thus, targeting TEM8 on tumor vasculature is useful for the selective blockade of cancer and other diseases dependent on pathological angiogenesis.

An exemplary TEM8 polynucleotide is provided in GenBank® Accession No. BC012074, and an exemplary TEM8 polypeptide is provided in GenBank® Accession No. AAH12074, both of which are incorporated by reference herein in their entirety.

### II. Chimeric Antigen Receptors

Genetic engineering of human T lymphocytes to express tumor-directed chimeric antigen receptors (CAR) can produce antitumor effector cells that bypass tumor immune escape mechanisms that are due to abnormalities in protein-antigen processing and presentation. Moreover, these transgenic receptors can be directed to tumor-associated antigens that are not protein-derived. In certain embodiments of the invention there are CTLs that are modified to comprise at least a CAR.

In particular cases, the cytotoxic T lymphocytes (CTLs) include a receptor that is chimeric, non-natural and engineered at least in part by the hand of man. In particular cases, the engineered CAR has one, two, three, four, or more components, and in some embodiments the one or more components facilitate targeting or binding of the T lymphocyte to the tumor antigen-comprising cancer cell. In specific embodiments, the CAR comprises an antibody for the tumor antigen, part or all of a cytoplasmic signaling domain, and/or part or all of one or more co-stimulatory molecules, for example endodomains of co-stimulatory molecules. In specific embodiments, the antibody is a single-chain variable fragment (scFv). In certain aspects the antibody is directed at target antigens on the cell surface of tumor vasculature, such as TEM1 and/or TEM8, for example. In certain embodiments, a cytoplasmic signaling domain, such as those derived from the T cell receptor ζ-chain, is employed as at least part of the chimeric receptor in order to produce stimulatory signals for T lymphocyte proliferation and effector function following engagement of the chimeric receptor with the target antigen. Examples would include, but are not limited to, endodomains from co-stimulatory molecules such as CD28, CD27, 4-1BB, and OX40. In particular embodiments, co-stimulatory molecules are employed to enhance the activation, proliferation, and cytotoxicity of T cells produced by the CAR after antigen engagement. In specific embodiments, the co-stimulatory molecules are CD28, CD27, OX40, and 4-1BB. T-cells can also be further genetically modified to enhance their function. Examples, but not limited to, include the transgenic expression of cytokines (e.g. IL2, IL7, IL15), silencing of negative regulators (for example SHP-1, FAS), chemokine receptors (e.g. CXCR2, CCR2b), dominant negative receptors (e.g. dominant negative TGFβRII), and/or so called ‘signal converters’ that convert a negative into a positive signal (e.g. IL4/IL2 chimeric cytokine receptor, IL4/IL7 chimeric cytokine receptor, or TGFβRII/TLR chimeric receptor).

In a particular embodiment, the components of the CAR in the polynucleotide that encodes it are in a particular order so that the expressed CAR protein has the corresponding domains in a particular order. For example, in particular embodiments the transmembrane domain is configured between the antibody domain and the endodomain. In specific embodiments, the order of the domains in the encoded CAR protein is N-terminal-antibody-transmembrane domain-endodomain-C terminal, although in certain cases the order of the domains in the encoded CAR protein is N-terminal-endodomain-transmembrane domain-antibody-C terminal. Of course, other domains may be inserted within this configuration, with care being taken to place it on the appropriate side of the transmembrane domain to be located inside the cell or on the surface of the cell. Those domains that need to be intracellular need to be on the flank of the transmembrane domain in the protein that the endodomain is located, for example. Those domains that need to be extracellular need to be on the flank of the transmembrane domain in the protein that the antibody is located.

The CAR may be first generation (CAR that includes the intracellular domain from the CD3 ζ-chain), second generation (CAR that also includes iintracellular signaling domains from various costimulatory protein receptors (e.g., CD28, 41BB, ICOS)), or third generation (CAR in which there are multiple signaling domains such as when signaling is provided by CD3-ζ together with co-stimulation provided by CD28 and a member of the tumor necrosis factor receptor superfamily, such as 4-1BB or OX40), for example. The CAR may be specific for TEM1, TEM8, or both (or a cell contains at least one of each). Cells expressing TEM1-specific CARs, TEM8-specific CARs, or both, may additionally express one or more additional CARs, e.g., CARs that bind to a TAA or TSA, e.g., such as those specific for EphA2, HER2, GD2, Glypican-3, 5T4, 8H9, αᵥβ₆ integrin, B cell maturation antigen (BCMA) B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor α, GD2, GD3, HLA-AI MAGE A1, HLA-A2, IL11Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, ROR1, Sp17, SURVIVIN, TAG72, TEM1, TEM8, VEGRR2, carcinoembryonic antigen, HMW-MAA, VEGF receptors, and/or other exemplary antigens that are present with in the extracelluar matrix of tumors, such as oncofetal variants of fibronectin, tenascin, or necrotic regions of tumors and other tumor-associated antigens or actionable mutations that are identified through genomic analysis and or differential expression studies of tumors, for example.

In certain embodiments, a CAR that directs an immune cell to TEM1 or TEM8 comprises (1) an extracellular antigen-binding domain that binds to TEM1 or TEM8, and (2) an intracellular domain that comprises a primary signaling moiety, e.g., a CD3ζ chain, that provides a primary T cell activation signal, and optionally a costimulatory moiety, e.g., a CD28 polypeptide and/or a 4-1BB (CD137) polypeptide. In certain specific embodiments, the T cell comprises a first CAR that provides a primary T cell activation signal, and a second CAR that provides a costimulatory signal. In various specific embodiments, the first CAR binds to TEM1 and provides a primary T cell activation signal, and the second CAR binds to TEM8 and provides a costimulatory signal; the first CAR binds to TEM8 and provides a primary T cell activation signal, and the second CAR binds TEM1 and provides a costimulatory signal; the first CAR binds TEM1 and provides a primary T cell activation signal and the second CAR binds a tumor associated antigen (TAA) or tumor specific antigen (TSA) and provides a costimulatory signal; the first CAR binds a TAA or TSA and provides a primary T cell activation signal and the second CAR binds TEM1 and provides a costimulatory signal; the first CAR binds TEM8 and provides a primary T cell activation signal and the second CAR binds a TAA or TSA and provides a costimulatory signal; the first CAR binds a TAA or TSA and provides a primary T cell activation signal and the second CAR binds TEM8 and provides a costimulatory signal.

In particular cases, the CAR is specific for TEM1 or TEM8, and in certain embodiments, the present invention provides chimeric T cells specific for TEM1 or TEM8 by joining an extracellular antigen-binding domain derived from the TEM1- or TEM8-specific antibody to cytoplasmic signaling domains derived from the T-cell receptor ζ-chain, with the endodomains of the exemplary costimulatory molecules CD28 and OX40, for examples. This CAR is expressed in human T cells and the targeting of TEM1 or TEM8-positive cancers is encompassed in the invention. In some cases, the same cell comprises a CAR specific for TEM1 and a CAR specific for TEM8. In some cases, the same cell comprises a CAR specific for TEM1 and a CAR specific for another antigen that may or may not be present on tumor vasculature. In some cases, the same cell comprises a CAR specific for TEM8 and a CAR specific for another antigen that may or may not be present on tumor vasculature.

In particular embodiments, a CAR specific for TEM1 refers to a CAR having a scFv antibody that recognizes TEM1. Although in some embodiments the TEM1 scFv is of any kind, in other embodiments the scFv is not the PTA-7554 TEM1 antibody in US 2010/0260769, US 2013/0078242, US 2011/0033455, US 2010/0021454, US 2008/0248034, U.S. Pat. No. 7,615,372, U.S. Pat. No. 7,807,382, U.S. Pat. No. 8,389,691, or U.S. Pat. No. 8,524,237, the disclosures of each of which are incorporated by reference herein in their entirety. In specific embodiments, the scFv is anti-TEM1 biobody-78.

In specific embodiments, a representative TEM8 CAR is in SEQ ID NO:1, and a representative TEM1 CAR is in SEQ ID NO:2.

III. Host Cells Expressing TEM1 and/or TEM8 CARs

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a eukaryotic cell that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells which do not contain a recombinantly introduced nucleic acid. In embodiments of the invention, a host cell is a T cell, including a cytotoxic T-cell (also known as TC, Cytotoxic T Lymphocyte, CTL, T-Killer cell, cytolytic T cell, CD8+ T-cells, CD4+ T-cells, or killer T-cells); NK cells and NKT cells are also encompassed in the invention.

In one aspect, provided herein is a cell that has been genetically engineered to express one or more CARs. In certain embodiments, the genetically engineered cell is, e.g., a T lymphocyte (T-cell), a natural killer (NK) T-cell, or an NK cell. In certain other embodiments, the genetically engineered cell is a non-immune cell, e.g., a mesenchymal stem cell (MSC), a neuronal stem cell, a hematopoietic stem cell, an induced pluripotent stem cell (iPS cell), or an embryonic stem cell, for example. In specific embodiments, the cell also comprises an engineered CAR or any other genetic modification that may enhance its function. In a particular embodiment, the antigen binding domain of the CAR binds TEM1 or TEM8, although in certain embodiments the antigen binding domain of the CAR recognizes a different target antigen.

In certain embodiments, it is contemplated that RNAs or proteinaceous sequences may be co expressed with other selected RNAs or proteinaceous sequences in the same cell, such as the same CTL. Co expression may be achieved by co transfecting the CTL with two or more distinct recombinant vectors. Alternatively, a single recombinant vector may be constructed to include multiple distinct coding regions for RNAs, which could then be expressed in CTLs transfected with the single vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

The cells can be autologous cells, syngeneic cells, allogenic cells and even in some cases, xenogeneic cells.

In many situations one may wish to be able to kill the genetically engineered T-cells, where one wishes to terminate the treatment, the cells become neoplastic, in research where the absence of the cells after their presence is of interest, or other purpose. For this purpose one can provide for the expression of certain gene products in which one can kill the engineered cells under controlled conditions, such as inducible suicide genes. Such suicide genes are known in the art, e.g., the iCaspase9 system in which a modified form of caspase 9 is dimerizable with a small molecule, e.g., AP1903. See, e.g., Straathof et al., Blood 105:4247-4254 (2005).

It is further envisaged that the pharmaceutical composition of the disclosure comprises a host cell transformed or transfected with a vector defined herein. The host cell may be produced by introducing at least one of the above described vectors or at least one of the above described nucleic acid molecules into the host cell. The presence of the at least one vector or at least one nucleic acid molecule in the host may mediate the expression of a gene encoding the above described be specific single chain antibody constructs.

The described nucleic acid molecule or vector that is introduced in the host cell may either integrate into the genome of the host or it may be maintained extrachromosomally.

The host cell can be any prokaryote or eukaryotic cell, but in specific embodiments it is a eukaryotic cell. In specific embodiments, the host cell is a bacterium, an insect, fungal, plant or animal cell. It is particularly envisaged that the recited host may be a mammalian cell, more preferably a human cell or human cell line. Particularly preferred host cells comprise immune cells, CHO cells, COS cells, myeloma cell lines like SP2/0 or NS/0.

The pharmaceutical composition of the disclosure may also comprise a proteinaceous compound capable of providing an activation signal for immune effector cells useful for cell proliferation or cell stimulation. In the light of the present disclosure, the “proteinaceous compounds” providing an activation signal for immune effector cells may be, e.g. a further activation signal for T-cells (e.g. a further costimulatory molecule: molecules of the B7-family, OX40 L, 4-1BBL), or a further cytokine: interleukin (e.g. IL-2, IL-7, or IL-15), or an NKG-2D engaging compound. The proteinaceous compound may also provide an activation signal for immune effector cell which is a non-T-cell. Examples for immune effector cells which are non-T-cells comprise, inter alia, NK cells, or NKT-cells.

One embodiment relates to a process for the production of a composition of the disclosure, the process comprising culturing a host cell defined herein above under conditions allowing the expression of the construct, and the cell or a plurality of cells is provided to the individual.

The conditions for the culturing of cells harboring an expression construct that allows the expression of the CAR molecules are known in the art, as are procedures for the purification/recovery of the constructs when desired.

In one embodiment, the host cell is a genetically engineered T-cell (e.g., cytotoxic T lymphocyte) comprising a CAR and in particular embodiments the cell further comprises an engineered TCR. Naturally occurring T-cell receptors comprise two subunits, an α-subunit and a β-subunit, each of which is a unique protein produced by recombination event in each T-cell's genome. Libraries of TCRs may be screened for their selectivity to particular target antigens. An “engineered TCR” refers to a natural TCR, which has a high-avidity and reactivity toward target antigens that is selected, cloned, and/or subsequently introduced into a population of T-cells used for adoptive immunotherapy. In contrast to engineered TCRs, CARs are engineered to bind target antigens in an MHC independent manner.

### IV. Pharmaceutical Compositions

Provided herein are pharmaceutical compositions comprising the genetically engineered immune cells, e.g., genetically engineered CAR T cells.

In accordance with this disclosure, the term “pharmaceutical composition” relates to a composition for administration to an individual. In a preferred embodiment, the pharmaceutical composition comprises a composition for parenteral, transdermal, intraluminal, intra-arterial, intrathecal or intravenous administration or for direct injection into a cancer. It is in particular envisaged that said pharmaceutical composition is administered to the individual via infusion or injection. Administration of the suitable compositions may be effected by different ways, e.g., by intravenous, subcutaneous, intraperitoneal, intramuscular, topical or intradermal administration.

The pharmaceutical composition of the present disclosure may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. An example of a dosage for administration might be in the range of 0.24 μg to 48 mg, preferably 0.24 μg to 24 mg, more preferably 0.24 μg to 2.4 mg, even more preferably 0.24 μg to 1.2 mg and most preferably 0.24 μg to 240 mg units per kilogram of body weight per day. Particularly preferred dosages are recited herein below. Progress can be monitored by periodic assessment.

The CAR cell compositions of the disclosure may be administered locally or systemically. Administration will generally be parenteral, e.g., intravenous; DNA may also be administered directly to the target site, e.g., by biolistic delivery to an internal or external target site or by catheter to a site in an artery. In a preferred embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. It is envisaged that the pharmaceutical composition of the disclosure might comprise, in addition to the proteinaceous bispecific single chain antibody constructs or nucleic acid molecules or vectors encoding the same (as described in this disclosure), further biologically active agents, depending on the intended use of the pharmaceutical composition.

Any of the compositions described herein may be comprised in a kit. In a non-limiting example, one or more cells for use in cell therapy and/or the reagents to generate one or more cells for use in cell therapy that harbors recombinant expression vectors may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments, cells that are to be used for cell therapy are provided in a kit, and in some cases the cells are essentially the sole component of the kit. The kit may comprise reagents and materials to make the desired cell. In specific embodiments, the reagents and materials include primers for amplifying desired sequences, nucleotides, suitable buffers or buffer reagents, salt, and so forth, and in some cases the reagents include vectors and/or DNA that encodes a CAR molecule as described herein and/or regulatory elements therefor.

In particular embodiments, there are one or more apparatuses in the kit suitable for extracting one or more samples from an individual. The apparatus may be a syringe, scalpel, and so forth.

In some cases, the kit, in addition to cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

### V. Therapeutic Uses of CARs and Host T-cells Comprising CARs

In various embodiments CAR constructs, nucleic acid sequences, vectors, host cells, as contemplated herein and/or pharmaceutical compositions comprising the same are used for the prevention, treatment or amelioration of a cancerous disease, such as a tumorous disease, or any disease wherein vasculature is a detriment. In particular embodiments, the pharmaceutical composition of the present disclosure may be particularly useful in preventing, ameliorating and/or treating cancer, including cancer having solid tumors, for example.

In particular embodiments, provided herein is a method of treating an individual for cancer, comprising the step of providing a therapeutically effective amount of a plurality of any of cells of the disclosure to the individual. In certain aspects, the cancer is a solid tumor, and the tumor may be of any size, but in specific embodiments, the solid tumors are about 2 mm or greater in diameter. In certain aspects, the method further comprises the step of providing a therapeutically effective amount of an additional cancer therapy to the individual.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

In particular embodiments, the present invention contemplates, in part, cells, CAR constructs, nucleic acid molecules and vectors that can administered either alone or in any combination using standard vectors and/or gene delivery systems, and in at least some aspects, together with a pharmaceutically acceptable carrier or excipient. In certain embodiments, subsequent to administration, said nucleic acid molecules or vectors may be stably integrated into the genome of the subject.

In specific embodiments, viral vectors may be used that are specific for certain cells or tissues and persist in said cells. Suitable pharmaceutical carriers and excipients are well known in the art. The compositions prepared according to the disclosure can be used for the prevention or treatment or delaying the above identified diseases.

Furthermore, the disclosure relates to a method for the prevention, treatment or amelioration of a tumorous disease comprising the step of administering to a subject or individual in the need thereof an effective amount of immune cells, e.g., T cells or cytotoxic T lymphocytes, harboring a TEM1 CAR or TEM8 CAR; a nucleic acid sequence encoding a TEM1 CAR, a TEM8 CAR or both; a vector comprising a nucleotide sequence encoding a TEM1 CAR or a TEM8 CAR or both, as described herein and/or produced by a process as described herein.

Possible indications for administration of the composition(s) of the exemplary CAR cells are cancerous diseases, including tumorous diseases, including breast, prostate, lung, and colon cancers or epithelial cancers/carcinomas such as breast cancer, colon cancer, prostate cancer, head and neck cancer, skin cancer, cancers of the genitourinary tract, e.g. ovarian cancer, endometrial cancer, cervical cancer and kidney cancer, lung cancer, gastric cancer, cancer of the small intestine, liver cancer, pancreatic cancer, gall bladder cancer, cancers of the bile duct, esophagus cancer, cancer of the salivary glands and cancer of the thyroid gland. The administration of the composition(s) of the disclosure is useful for all stages and types of cancer, including for minimal residual disease, early cancer, advanced cancer, and/or metastatic cancer and/or refractory cancer, for example, wherein the cancer is associated with pathogenic vascularization.

The disclosure further encompasses co-administration protocols with other compounds, e.g. bispecific antibody constructs, targeted toxins or other compounds, which act via immune cells. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

Particular doses for therapy may be determined using routine methods in the art. However, in specific embodiments, the T cells are delivered to an individual in need thereof once, although in some cases it is multiple times, including 2, 3, 4, 5, 6, or more times. When multiple doses are given, the span of time between doses may be of any suitable time, but in specific embodiments, it is weeks or months between the doses. The time between doses may vary in a single regimen. In particular embodiments, the time between doses is 2, 3, 4, 5, 6, 7, 8, 9, 10, or more weeks. In specific cases, it is between 4-8 or 6-8 weeks, for example. In specific embodiments, one regimen includes the following dose regimen:

| Dose Level | T cell Dose |
|---|---|
| 1 | 1 × 10⁶/m² |
| 2 | 3 × 10⁶/m² |
| 3 | 1 × 10⁷/m² |
| 4 | 3 × 10⁷/m² |
| 5 | 1 × 10⁸/m² |

In an alternative embodiment, the T cells are provided to the individual in the following dose regimen:

| Dose Level | CTL Dose |
|---|---|
| 1 | 1 × 10⁶/m² |
| 2 | 1 × 10⁷/m² |
| 3 | 1 × 10⁸/m² |

Embodiments relate to a kit comprising a bispecific single chain antibody construct as defined above, a nucleic acid sequence as defined above, a vector as defined above and/or a host as defined above. It is also contemplated that the kit of this disclosure comprises a pharmaceutical composition as described herein above, either alone or in combination with further medicaments to be administered to an individual in need of medical treatment or intervention.

In particular embodiments, there are pharmaceutical compositions that comprise cells that express TEM1-specific or TEM-8 specific CARs. An effective amount of the cells are given to an individual in need thereof.

By way of illustration, cancer patients or patients susceptible to cancer or suspected of having cancer may be treated as follows. T-cells modified as described herein may be administered to the patient and retained for extended periods of time. The individual may receive one or more administrations of the cells. In some embodiments, the genetically engineered cells are encapsulated to inhibit immune recognition and placed at the site of the tumor.

In particular cases the individual is provided with therapeutic T-cells engineered to comprise a CAR specific for TEM1 and/or a CAR specific for TEM8. The cells may be delivered at the same time or at different times, wherein the CARs for TEM and TEM8 are in separate cells. The cells may be delivered in the same or separate formulations. The cells may be provided to the individual in separate delivery routes. The cells may be delivered by injection at a tumor site or intravenously or orally, for example. Routine delivery routes for such compositions are known in the art.

Expression vectors that encode the TEM1 and/or TEM8 CARs can be introduced as one or more DNA molecules or constructs, where there may be at least one marker that will allow for selection of host cells that contain the construct(s). The constructs can be prepared in conventional ways, where the genes and regulatory regions may be isolated, as appropriate, ligated, cloned in an appropriate cloning host, analyzed by restriction or sequencing, or other convenient means. Particularly, using PCR, individual fragments including all or portions of a functional unit may be isolated, where one or more mutations may be introduced using “primer repair”, ligation, in vitro mutagenesis, etc., as appropriate. The construct(s) once completed and demonstrated to have the appropriate sequences may then be introduced into the CTL by any convenient means. The constructs may be integrated and packaged into non-replicating, defective viral genomes like Adenovirus, Adeno-associated virus (AAV), or Herpes simplex virus (HSV) or others, including retroviral vectors, for infection or transduction into cells. The constructs may include viral sequences for transfection, if desired. Alternatively, the construct may be introduced by fusion, electroporation, biolistics, transfection, lipofection, or the like. The host cells may be grown and expanded in culture before introduction of the construct(s), followed by the appropriate treatment for introduction of the construct(s) and integration of the construct(s). The cells are then expanded and screened by virtue of a marker present in the construct. Various markers that may be used successfully include hprt, neomycin resistance, thymidine kinase, hygromycin resistance, etc.

In some instances, one may have a target site for homologous recombination, where it is desired that a construct be integrated at a particular locus. For example,) can knock-out an endogenous gene and replace it (at the same locus or elsewhere) with the gene encoded for by the construct using materials and methods as are known in the art for homologous recombination. For homologous recombination, one may use either .OMEGA. or O-vectors. See, for example, Thomas and Capecchi, Cell (1987) 51, 503-512; Mansour, et al., Nature (1988) 336, 348-352; and Joyner, et al., Nature (1989) 338, 153-156.

The constructs may be introduced as a single DNA molecule encoding at least the TEM1- and/or TEM8-specific CAR and optionally another gene, or different DNA molecules having one or more genes. The constructs may be introduced simultaneously or consecutively, each with the same or different markers.

Vectors containing useful elements such as bacterial or yeast origins of replication, selectable and/or amplifiable markers, promoter/enhancer elements for expression in prokaryotes or eukaryotes, etc. that may be used to prepare stocks of construct DNAs and for carrying out transfections are well known in the art, and many are commercially available.

The exemplary T cells that have been engineered to include the TEM1 or TEM8 CAR construct(s) are then grown in culture under selective conditions and cells that are selected as having the construct may then be expanded and further analyzed, using, for example; the polymerase chain reaction for determining the presence of the construct in the host cells. Once the engineered host cells have been identified, they may then be used as planned, e.g. expanded in culture or introduced into a host organism.

Depending upon the nature of the cells, the cells may be introduced into a host organism, e.g. a mammal, in a wide variety of ways. The cells may be introduced at the site of the tumor, in specific embodiments, although in alternative embodiments the cells hone to the cancer or are modified to hone to the cancer. The number of cells that are employed will depend upon a number of circumstances, the purpose for the introduction, the lifetime of the cells, the protocol to be used, for example, the number of administrations, the ability of the cells to multiply, the stability of the recombinant construct, and the like. The cells may be applied as a dispersion, generally being injected at or near the site of interest. The cells may be in a physiologically-acceptable medium.

The DNA introduction need not result in integration in every case. In some situations, transient maintenance of the DNA introduced may be sufficient. In this way, one could have a short term effect, where cells could be introduced into the host and then turned on after a predetermined time, for example, after the cells have been able to home to a particular site.

The cells may be administered as desired. Depending upon the response desired, the manner of administration, the life of the cells, the number of cells present, various protocols may be employed. The number of administrations will depend upon the factors described above at least in part.

It should be appreciated that the system is subject to many variables, such as the cellular response to the ligand, the efficiency of expression and, as appropriate, the level of secretion, the activity of the expression product, the particular need of the patient, which may vary with time and circumstances, the rate of loss of the cellular activity as a result of loss of cells or expression activity of individual cells, and the like. Therefore, it is expected that for each individual patient, even if there were universal cells which could be administered to the population at large, each patient would be monitored for the proper dosage for the individual, and such practices of monitoring a patient are routine in the art.

In another aspect, provided herein is a method of treating an individual having a tumor cell, comprising administering to the individual a therapeutically effective amount of cells expressing at least TEM1-specific CAR and/or TEM8-specific CAR. In a related aspect, provided herein is a method of treating an individual having a tumor cell, comprising administering to the individual a therapeutically effective amount of cells expressing at least TEM1-specific CAR and/or TEM8-specific CAR. In a specific embodiment, said administering results in a measurable decrease in the growth of the tumor in the individual. In another specific embodiment, said administering results in a measurable decrease in the size of the tumor in the individual. In various embodiments, the size or growth rate of a tumor may be determinable by, e.g., direct imaging (e.g., CT scan, MRI, PET scan or the like), fluorescent imaging, tissue biopsy, and/or evaluation of relevant physiological markers (e.g., PSA levels for prostate cancer; HCG levels for choriocarcinoma, and the like). In specific embodiments of the invention, the individual has a high level of an antigen that is correlated to poor prognosis. In some embodiments, the individual is provided with an additional cancer therapy, such as surgery, radiation, chemotherapy, hormone therapy, immunotherapy, or a combination thereof.

Embodiments relate to a kit comprising cells as defined herein, CAR constructs as defined herein, a nucleic acid sequence as defined herein, and/or a vector as defined herein. It is also contemplated that the kit of this disclosure comprises a pharmaceutical composition as described herein above, either alone or in combination with further medicaments to be administered to an individual in need of medical treatment or intervention.

### VI. Polynucleotide Encoding CARs

The present disclosure also encompasses a composition comprising a nucleic acid sequence encoding a CAR as defined herein and cells harboring the nucleic acid sequence. The nucleic acid molecule is a recombinant nucleic acid molecule, in particular aspects and may be synthetic. It may comprise DNA, RNA as well as PNA (peptide nucleic acid) and it may be a hybrid thereof.

It is evident to the person skilled in the art that one or more regulatory sequences may be added to the nucleic acid molecule comprised in the composition of the disclosure. For example, promoters, transcriptional enhancers and/or sequences that allow for induced expression of the polynucleotide of the disclosure may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62), or a dexamethasone-inducible gene expression system as described, e.g. by Crook (1989) EMBO J. 8, 513-519.

Furthermore, it is envisaged for further purposes that nucleic acid molecules may contain, for example, thioester bonds and/or nucleotide analogues. The modifications may be useful for the stabilization of the nucleic acid molecule against endo- and/or exonucleases in the cell. The nucleic acid molecules may be transcribed by an appropriate vector comprising a chimeric gene that allows for the transcription of said nucleic acid molecule in the cell. In this respect, it is also to be understood that such polynucleotides can be used for “gene targeting” or “gene therapeutic” approaches. In another embodiment the nucleic acid molecules are labeled. Methods for the detection of nucleic acids are well known in the art, e.g., Southern and Northern blotting, PCR or primer extension. This embodiment may be useful for screening methods for verifying successful introduction of the nucleic acid molecules described above during gene therapy approaches.

The nucleic acid molecule(s) may be a recombinantly produced chimeric nucleic acid molecule comprising any of the aforementioned nucleic acid molecules either alone or in combination. In specific aspects, the nucleic acid molecule is part of a vector.

The present disclosure therefore also relates to a composition comprising a vector comprising the nucleic acid molecule described in the present disclosure.

Many suitable vectors are known to those skilled in molecular biology, the choice of which would depend on the function desired and include plasmids, cosmids, viruses, bacteriophages and other vectors used conventionally in genetic engineering. Methods that are well known to those skilled in the art can be used to construct various plasmids and vectors; see, for example, the techniques described in Sambrook et al. (1989) and Ausubel, Current Protocols in Molecular Biology, Green Publishing Associates and Wiley Interscience, N.Y. (1989), (1994). Alternatively, the polynucleotides and vectors of the disclosure can be reconstituted into liposomes for delivery to target cells. A cloning vector may be used to isolate individual sequences of DNA. Relevant sequences can be transferred into expression vectors where expression of a particular polypeptide is required. Typical cloning vectors include pBluescript SK, pGEM, pUC9, pBR322 and pGBT9. Typical expression vectors include pTRE, pCAL-n-EK, pESP-1, pOP13CAT.

In specific embodiments, there is a vector that comprises a nucleic acid sequence that is a regulatory sequence operably linked to the nucleic acid sequence encoding a CAR construct defined herein. Such regulatory sequences (control elements) are known to the artisan and may include a promoter, a splice cassette, translation initiation codon, translation and insertion site for introducing an insert into the vector. In specific embodiments, the nucleic acid molecule is operatively linked to said expression control sequences allowing expression in eukaryotic or prokaryotic cells.

It is envisaged that a vector is an expression vector comprising the nucleic acid molecule encoding a CAR construct defined herein. In specific aspects, the vector is a viral vector, such as a lentiviral vector. Lentiviral vectors are commercially available, including from Clontech (Mountain View, Calif.) or GeneCopoeia (Rockville, Md.), for example.

The term “regulatory sequence” refers to DNA sequences that are necessary to effect the expression of coding sequences to which they are ligated. The nature of such control sequences differs depending upon the host organism. In prokaryotes, control sequences generally include promoters, ribosomal binding sites, and terminators. In eukaryotes generally control sequences include promoters, terminators and, in some instances, enhancers, transactivators or transcription factors. The term “control sequence” is intended to include, at a minimum, all components the presence of which are necessary for expression, and may also include additional advantageous components.

The term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences. In case the control sequence is a promoter, it is obvious for a skilled person that double-stranded nucleic acid is preferably used.

Thus, the recited vector is an expression vector, in certain embodiments. An “expression vector” is a construct that can be used to transform a selected host and provides for expression of a coding sequence in the selected host. Expression vectors can for instance be cloning vectors, binary vectors or integrating vectors. Expression comprises transcription of the nucleic acid molecule preferably into a translatable mRNA. Regulatory elements ensuring expression in prokaryotes and/or eukaryotic cells are well known to those skilled in the art. In the case of eukaryotic cells they comprise normally promoters ensuring initiation of transcription and optionally poly-A signals ensuring termination of transcription and stabilization of the transcript. Possible regulatory elements permitting expression in prokaryotic host cells comprise, e.g., the PL, lac, trp or tac promoter in E. coli, and examples of regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells.

Beside elements that are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the recited nucleic acid sequence and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an N-terminal identification peptide imparting desired characteristics, e.g., stabilization or simplified purification of expressed recombinant product; see supra. In this context, suitable expression vectors are known in the art such as Okayama-Berg cDNA expression vector pcDV1 (Pharmacia), pEF-Neo, pCDM8, pRc/CMV, pcDNA1, pcDNA3 (Invitrogen), pEF-DHFR and pEF-ADA, (Raum et al. Cancer Immunol Immunother (2001) 50(3), 141-150) or pSPORT1 (GIBCO BRL).

In some embodiments, the expression control sequences are eukaryotic promoter systems in vectors capable of transforming of transfecting eukaryotic host cells, but control sequences for prokaryotic hosts may also be used. Once the vector has been incorporated into the appropriate host, the host is maintained under conditions suitable for high level expression of the nucleotide sequences, and as desired, the collection and purification of the polypeptide of the disclosure may follow.

Additional regulatory elements may include transcriptional as well as translational enhancers. Advantageously, the above-described vectors of the disclosure comprises a selectable and/or scorable marker. Selectable marker genes useful for the selection of transformed cells are well known to those skilled in the art and comprise, for example, antimetabolite resistance as the basis of selection for dhfr, which confers resistance to methotrexate (Reiss, Plant Physiol. (Life-Sci. Adv.) 13 (1994), 143-149); npt, which confers resistance to the aminoglycosides neomycin, kanamycin and paromycin (Herrera-Estrella, EMBO J. 2 (1983), 987-995) and hygro, which confers resistance to hygromycin (Marsh, Gene 32 (1984), 481-485). Additional selectable genes have been described, namely trpB, which allows cells to utilize indole in place of tryptophan; hisD, which allows cells to utilize histinol in place of histidine (Hartman, Proc. Natl. Acad. Sci. USA 85 (1988), 8047); mannose-6-phosphate isomerase which allows cells to utilize mannose (WO 94/20627) and ODC (ornithine decarboxylase) which confers resistance to the ornithine decarboxylase inhibitor, 2-(difluoromethyl)-DL-ornithine, DFMO (McConlogue, 1987, In: Current Communications in Molecular Biology, Cold Spring Harbor Laboratory ed.) or deaminase from Aspergillus terreus that confers resistance to Blasticidin S (Tamura, Biosci. Biotechnol. Biochem. 59 (1995), 2336-2338).

Useful scorable markers are also known to those skilled in the art and are commercially available. Advantageously, said marker is a gene encoding luciferase (Giacomin, Pl. Sci. 116 (1996), 59-72; Scikantha, J. Bact. 178 (1996), 121), green fluorescent protein (Gerdes, FEBS Lett. 389 (1996), 44-47) or β-glucuronidase (Jefferson, EMBO J. 6 (1987), 3901-3907). This embodiment is particularly useful for simple and rapid screening of cells, tissues and organisms containing a recited vector.

As described above, the recited nucleic acid molecule can be used in a cell, alone, or as part of a vector to express the encoded polypeptide in cells. The nucleic acid molecules or vectors containing the DNA sequence(s) encoding any one of the CAR constructs described herein is introduced into the cells that in turn produce the polypeptide of interest. The recited nucleic acid molecules and vectors may be designed for direct introduction or for introduction via liposomes, or viral vectors (e.g., adenoviral, retroviral) into a cell. In certain embodiments, the cells are T-cells, CAR T-cells, NK cells, NKT-cells, MSCs, neuronal stem cells, or hematopoietic stem cells, for example.

In accordance with the above, the present disclosure relates to methods to derive vectors, particularly plasmids, cosmids, viruses and bacteriophages used conventionally in genetic engineering that comprise a nucleic acid molecule encoding the polypeptide sequence of a CAR defined herein. In certain cases, said vector is an expression vector and/or a gene transfer or targeting vector. Expression vectors derived from viruses such as retroviruses, vaccinia virus, adeno-associated virus, herpes viruses, or bovine papilloma virus, may be used for delivery of the recited polynucleotides or vector into targeted cell populations. Methods that are well known to those skilled in the art can be used to construct recombinant vectors; see, for example, the techniques described in Sambrook et al. (loc cit.), Ausubel (1989, loc cit.) or other standard text books. Alternatively, the recited nucleic acid molecules and vectors can be reconstituted into liposomes for delivery to target cells. The vectors containing the nucleic acid molecules of the disclosure can be transferred into the host cell by well-known methods, which vary depending on the type of cellular host. For example, calcium chloride transfection is commonly utilized for prokaryotic cells, whereas calcium phosphate treatment or electroporation may be used for other cellular hosts; see Sambrook, supra.

### VII. Vectors Generally

The disclosure encompasses immune cells that are engineered to harbor a CAR-expressing DNA polynucleotide, which in certain embodiments is a vector having an expression construct or referred to as an expression vector. The elements of a vector may be routinely selected in the art, although those vectors for the present disclosure are unique in their incorporation of a TEM1-specific CAR and/or a TEM8-specific CAR.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

A. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art.

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages, and these may be used in the invention.

Vectors can include a multiple cloning site (MCS), which is a nucleic acid region that contains multiple restriction enzyme sites, any of which can be used in conjunction with standard recombinant technology to digest the vector. “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

Splicing sites, termination signals, origins of replication, and selectable markers may also be employed.

B. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEMTM 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with 13 galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

C. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Components of the present invention may be a viral vector that encodes one or more CARs of the invention. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the cells of the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector, a nucleic acid (e.g., one encoding the desired sequence) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

D. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

E. Vector Delivery and Cell Transformation

Suitable methods for nucleic acid delivery for transfection or transformation of cells are known to one of ordinary skill in the art. Such methods include, but are not limited to, direct delivery of DNA such as by ex vivo transfection, by injection, and so forth. Through the application of techniques known in the art, cells may be stably or transiently transformed.

F. Ex Vivo Transformation

Methods for transfecting eukaryotic cells and tissues removed from an organism in an ex vivo setting are known to those of skill in the art. Thus, it is contemplated that cells or tissues may be removed and transfected ex vivo using nucleic acids of the present invention. In particular aspects, the transplanted cells or tissues may be placed into an organism. In preferred facets, a nucleic acid is expressed in the transplanted cells.

### VIII. Combination Therapy

In certain embodiments of the invention, methods of the present invention for clinical aspects are combined with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cancer cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that cell therapy could be used similarly in conjunction with chemotherapeutic, radiotherapeutic, or immunotherapeutic intervention, in addition to other pro-apoptotic or cell cycle regulating agents.

Alternatively, the present inventive therapy may precede or follow the other agent treatment by intervals ranging from minutes to weeks. In embodiments where the other agent and present invention are applied separately to the individual, one would generally ensure that a significant period of time did not expire between the time of each delivery, such that the agent and inventive therapy would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, present invention is “A” and the secondary agent, such as radio- or chemotherapy, is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

It is expected that the treatment cycles would be repeated as necessary. It also is contemplated that various standard therapies, as well as surgical intervention, may be applied in combination with the inventive cell therapy.

A. Chemotherapy

Cancer therapies also include a variety of combination therapies with both chemical and radiation based treatments. Combination anti-cancer agents include, for example, acivicin; aclarubicin; acodazole hydrochloride; acronine; adozelesin; aldesleukin; altretamine; ambomycin; ametantrone acetate; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estrarnustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride; 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone: didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; Erbitux, human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin: neridronic acid; nilutamide; nisamycin; nitric oxide modulators; nitroxide antioxidant; nitrullyn; oblimersen (GENASENSE®); O.sup.6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; velaresol; veramine; verdins; verteporfin; vinorelbine; vinxaltine; vitaxin; vorozole; zanoterone; zeniplatin; zilascorb; and zinostatin stimalamer, or any analog or derivative variant of the foregoing and also combinations thereof.

In specific embodiments, chemotherapy for the individual is employed in conjunction with the invention, for example before, during and/or after administration of the invention.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics generally rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy other than the inventive therapy described herein could thus be used as part of a combined therapy, in conjunction with the present cell therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

In certain embodiments, the immunotherapy is an antibody against a Notch pathway ligand or receptor, e.g., an antibody against DLL4, Notch1, Notch2/3, Fzd7, or Wnt. In certain other embodiments, the immunotherapy is an antibody against r-spondin (RSPO) 1, RSPO2, RSPO3 or RSPO4.

D. Genes

In yet another embodiment, the secondary treatment is a gene therapy in which a therapeutic polynucleotide is administered before, after, or at the same time as the present invention clinical embodiments. A variety of expression products are encompassed within the invention, including inducers of cellular proliferation, inhibitors of cellular proliferation, or regulators of programmed cell death.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### General Embodiments

CARs were designed that are specific for TEM1 and TEM8, two antigens that are preferentially expressed in the tumor vasculature. Specifically, CARs were constructed based on published sequences of TEM1- and TEM8-specific monoclonal antibodies. TEM1- and TEM8-specific T cells were generated by retroviral transduction, and the inventors have shown that TEM1- and TEM8-specific CAR T cells recognize TEM1- or TEM8-positive target cells:

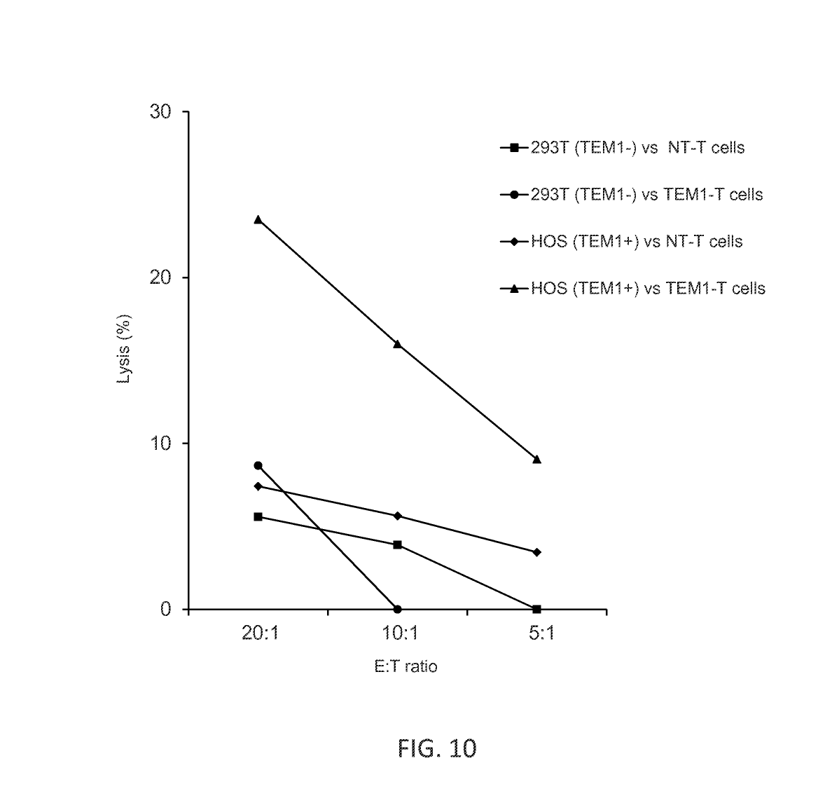

T cells expressing TEM1-specific CARs recognize TEM1-positive cancer cells. TEM1-specific CAR T cells (TEM1-CAR T cells) were co-cultured with TEM1-positive (HOS) and TEM1-negative (293T) target cells. After 24 hours media was collected for analysis. TEM1-CAR T cells recognized HOS cells in contrast to 293T cells as judged by the production of the proinflammatory cytokine IFNγ and IL-2 (FIG. 10).

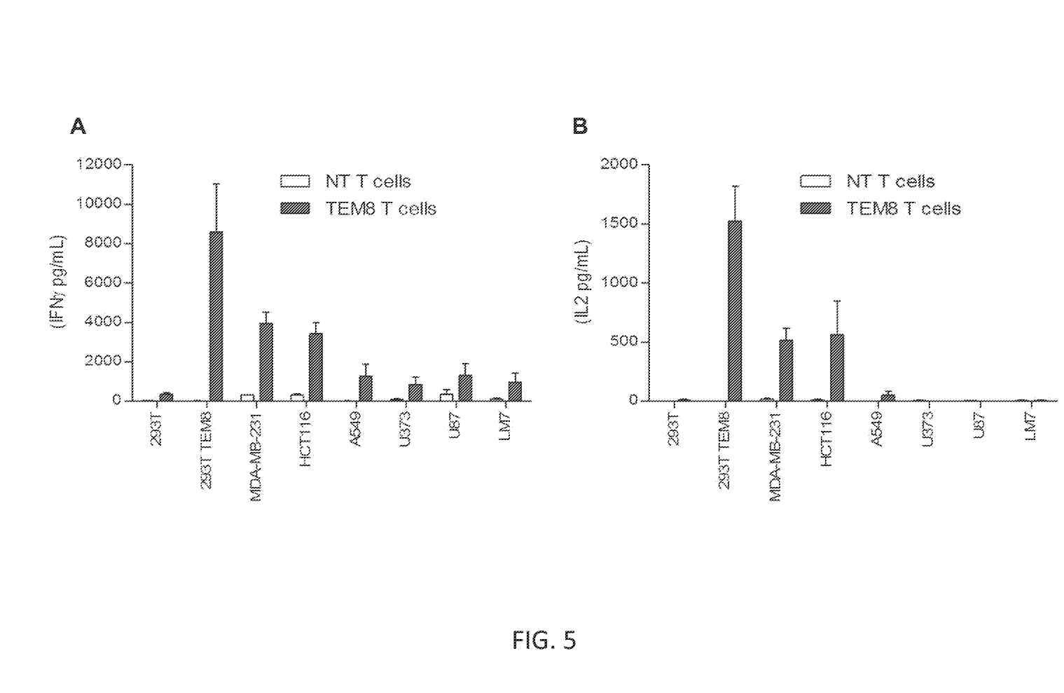

T cells expressing TEM8-specific CARs recognize TEM1-positive cancer cells. TEM8-specific CAR T cells (TEM8-CAR T cells) were co-cultured with TEM8-positive and TEM8-negative target cells. After 24 hours media was collected for analysis. TEM8-CAR T cells recognized TEM8-positive cells (293T-TEM8, MDA-MB231, HTT-116) as judged by the production of the proinflammatory cytokine IFNγ and IL-2 (FIG. 5). In contrast TEM8-negative cells (A549, U373, U87, LM7) did not induce IL-2 and only minimal IFNγ secretion (FIG. 5).

Embodiments of the present invention provide tumor vasculature-specific T cells by genetically modifying T cells with TEM1- and TEM8-specific CARs. This technology has broad application for the entire field of cancer therapy either as monotherapy or in combination with other treatment modalities for cancers that contain TEM1- and TEM8-positive tumor vasculature.

### Example 2

### TEM1-Specific T Cells

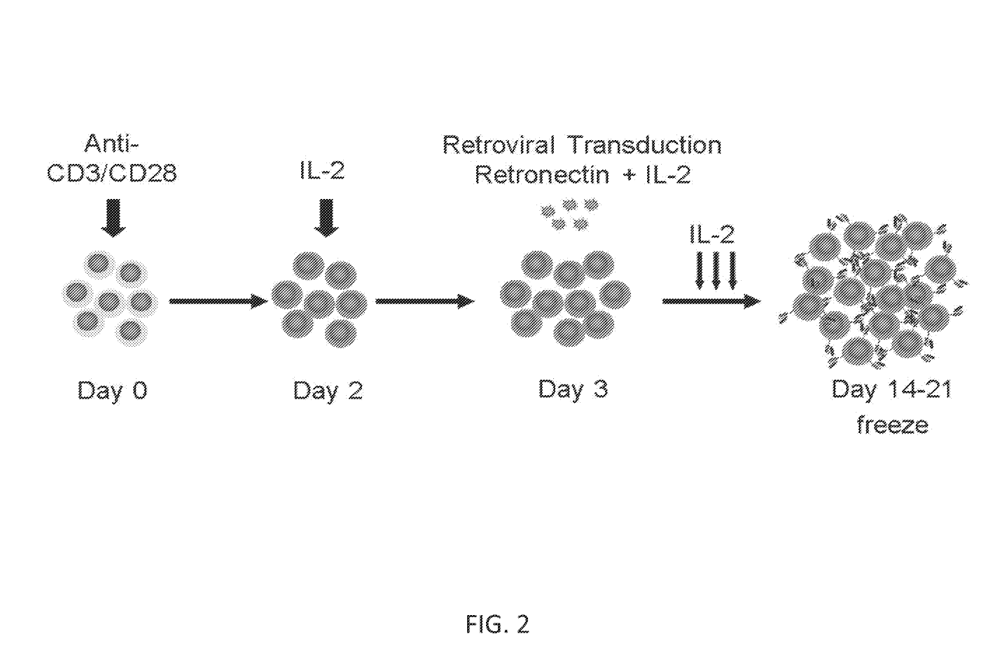

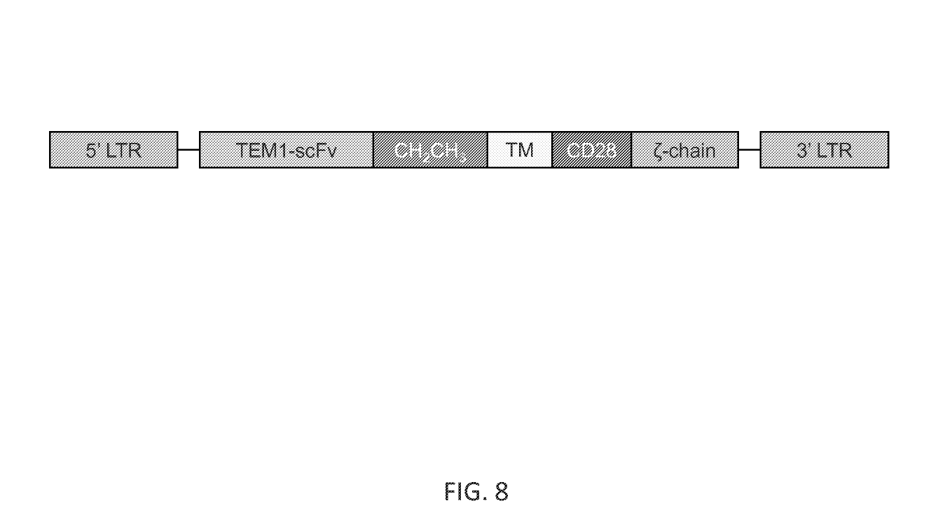

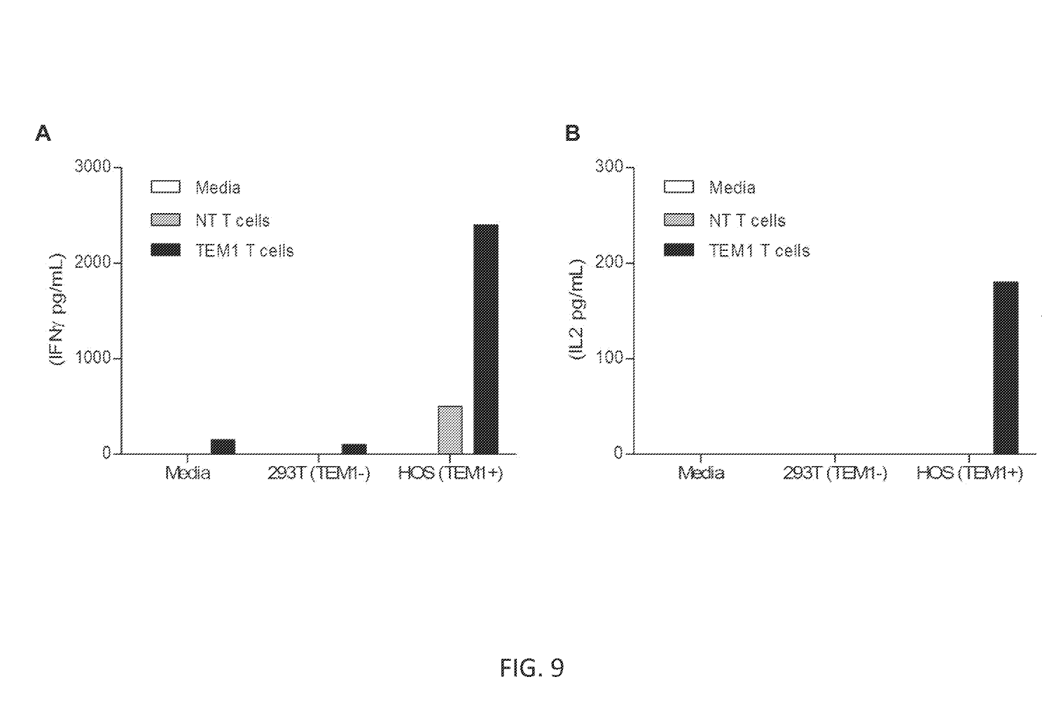

TEM1-specific T cells have been generated. FIG. 8 illustrates an exemplary TEM1-specific CAR and FIG. 2 is an exemplary method for generating TEM1-specific T cells. FIG. 9 demonstrates that TEM1-specific T cells produce IFN-γ and IL-2. FIG. 10 shows that TEM1-specific T cells kill TEM1-positive target cells in vitro.

### Example 3

### Generation of TEM1- and TEM8-Specific CAR T Cells

Adoptive T cell therapy is a tumor specific therapy in which blood is drawn from the patient, followed by isolation of their T cells. Their T cells are then engineered to be tumor specific and delivered back into the patient. T cells can be engineered to be tumor-specific by modifying the T cell to express a chimeric antigen receptor. CAR T cells combine the antigen-binding property of monoclonal antibodies with the signaling capacity and self-renewal of T cells. CAR-expressing T cells recognize and kill tumor cells in an MHC-unrestricted modality, so that target cell recognition by CAR-T cells is unaffected by some of the major mechanisms by which tumors avoid MHC-restricted T cell recognition.

In certain aspects, generation of CAR T cells may begin with the isolation of PBMCs or peripheral blood mononuclear cells from healthy donors and the addition of CD3/CD28 antibodies. IL2 is added to the OKT3/CD28 blasts to activate the T cells to proliferate. The exemplary retroviral supernatant is added with IL2 for transduction. Upon obtaining the sequence, a codon-optimized gene was synthesized, and it was cloned into a 3rd generation CAR SFG retroviral vector. RD114 retroviral particles were generated by transient transfection of 293T cells with an SFG retroviral vector. Specifically, three DNA plasmids encoding the gene of interest, a polymerase and a viral envelope are introduced into the packaging cell line 293T. Products of gene transcription and translation yield the necessary components to assemble a gutted retroviral vector that will transmit the gene of interest into the target cell line. GeneJuice transfection reagent may be employed in the generation of the cells.

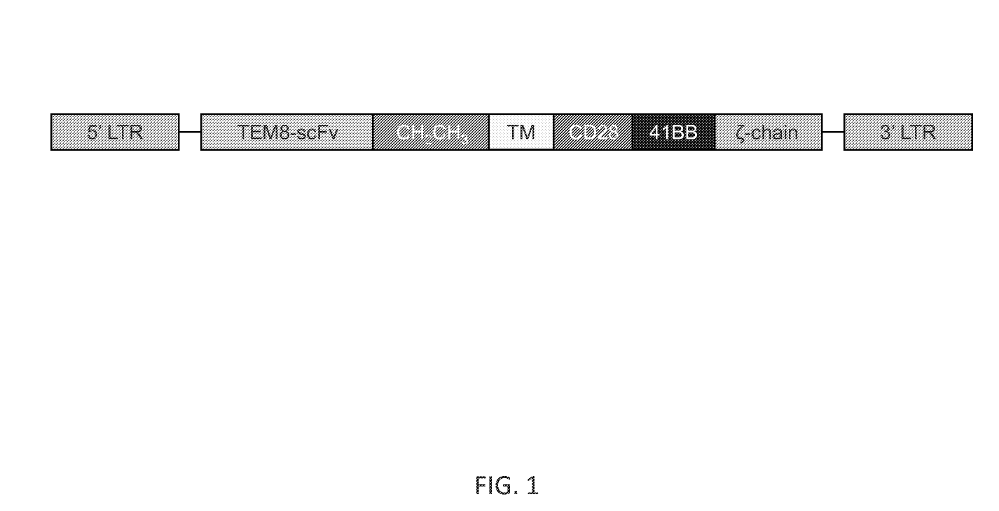

In other aspects, generation of CAR T cells may begin with the construction of retroviral vector encoding the CAR. Condon-optimized TEM1-specific and TEM8-specific scFvs were synthesized with a leader sequence and inserted into SFG retroviral vectors containing either a 2^{nd} generation CAR (for TEM1; FIG. 8) or 3^{rd} generation CAR (for TEM8; FIG. 1). Next RD114 retroviral particles were generated by transient transfection using transfection reagents, such as GeneJuice, of 293T cells with the SFG retroviral vector, a plasmid encoding the retroviral polymerase, and the viral envelope RD114. Other viral envelopes can also be used for example, but not limited to VSVG or GALV. To transduce human T cells, peripheral blood mononuclear cells (PBMC) are isolated from healthy donors or patients. These PBMCs are then activated with CD3/CD28 antibodies. Cytokines, such as IL2 or IL7/IL15, are also added to induce the proliferation of CD3/CD28-activated T cells. These activated T cells are then transduced with retroviral vectors and further expanded in the presence of cytokines. This exemplary method is summarized in FIG. 2.

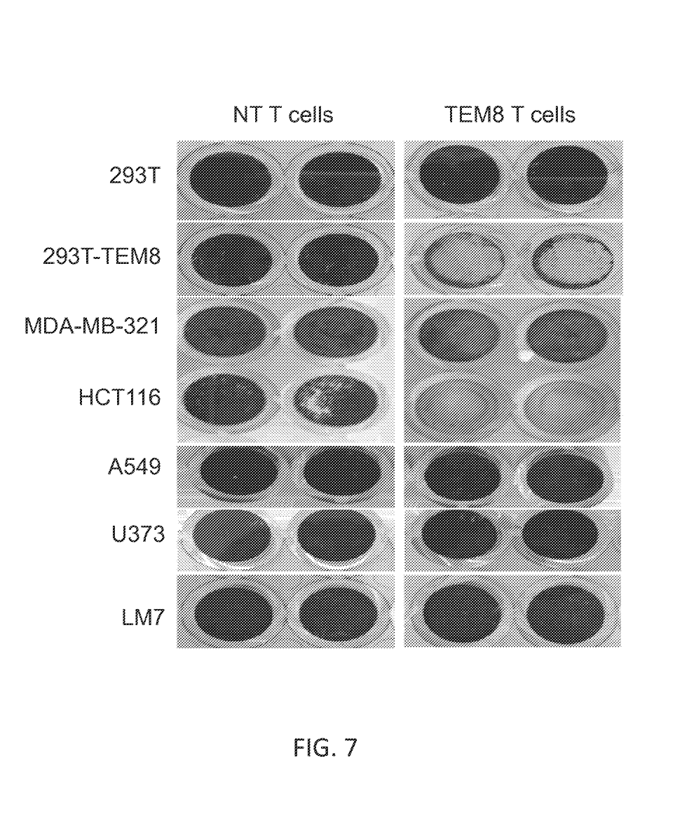

FIG. 7 shows that TEM8-CAR T cells recognize TEM8-positive cells in coculture assays in contrast to TEM8-negative cells as judged by cytokine production.

For retroviral transduction embodiments, one can treat plates overnight with OKT3 (CD3) and CD28 antibodies and then add PBMCs. The viral supernatant is then added. Retronectin enhances retroviral gene transduction in mammalian cells without toxicity.

FIG. 2 illustrates an exemplary method to generate CAR T-cells (Pule et al., Mol Ther 2005).

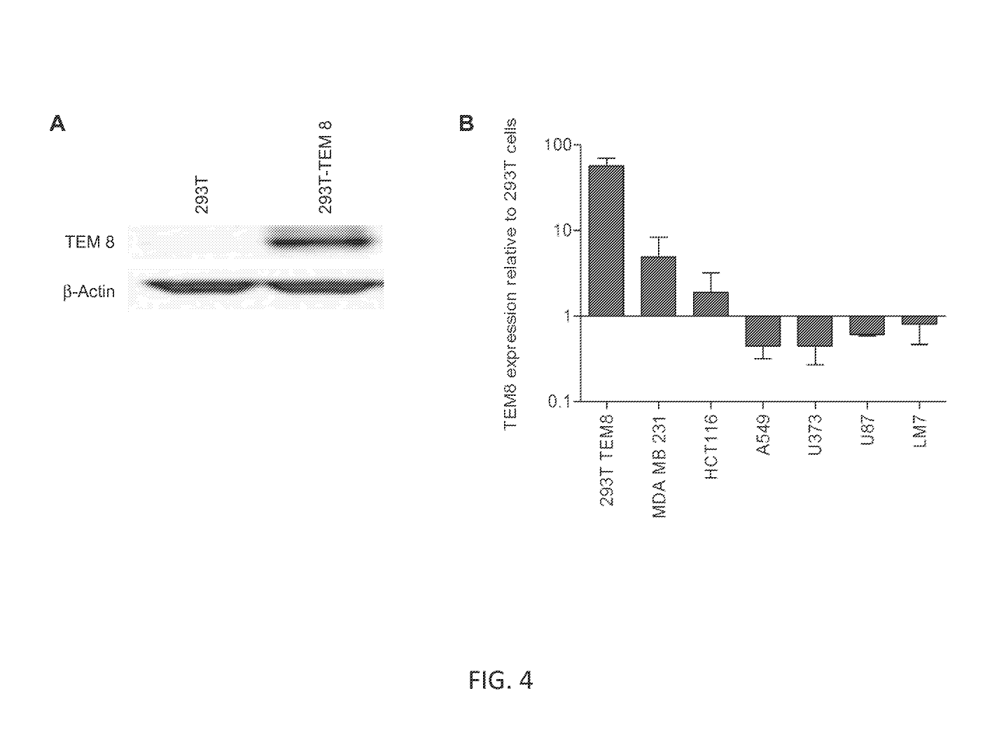

FIG. 4 demonstrates Western blot and qRT-PCR data of TEM8-positive and -negative cells.

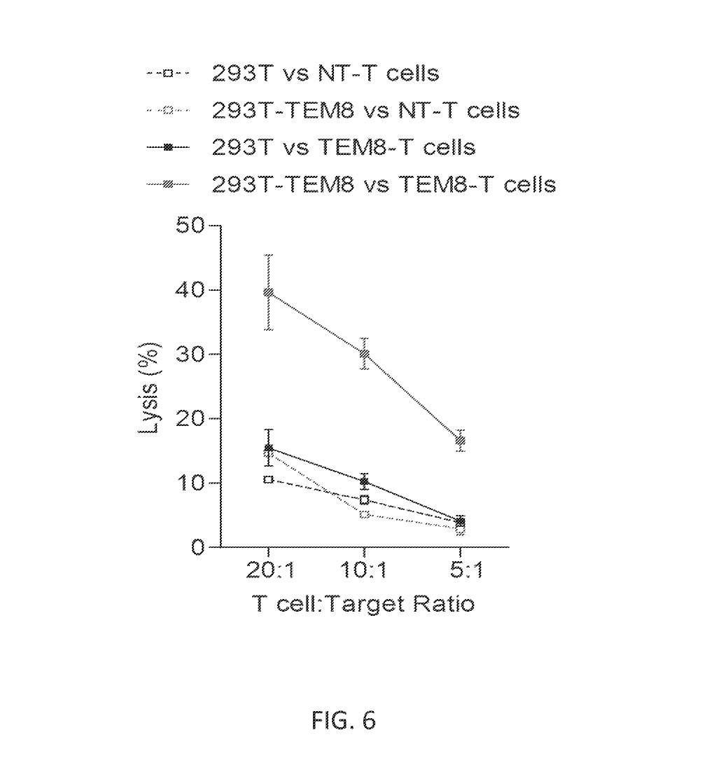

FIGS. 6 and 7 demonstrate that TEM 8-CAR T cells kill TEM8-positive cells while TEM8-negative cells are not killed.

FIG. 9 shows that TEM1-CAR T cells recognize TEM1-positive cells in coculture assays in contrast to TEM1-negative cells as judged by cytokine production.

### Example 4

### TEM8-Specific CARs and Use Thereof

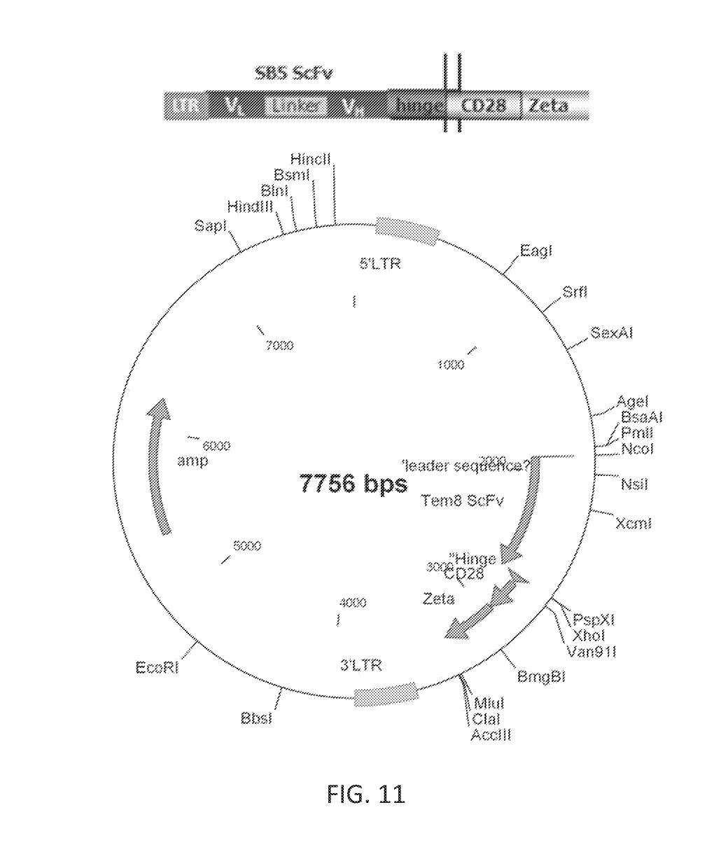

FIG. 11 illustrates an example of a TEM8 CAR. Shown therein is the exemplary TEM8 (SB5) CAR. The exodomain is derived from the SB5 scFv provided by Bradley Fletcher, University of Florida. Antibody origin; Bradley St Croix, NCI. FIG. 11A illustrates a corresponding example of a transgene, and FIG. 11B illustrates an example of a corresponding plasmid.

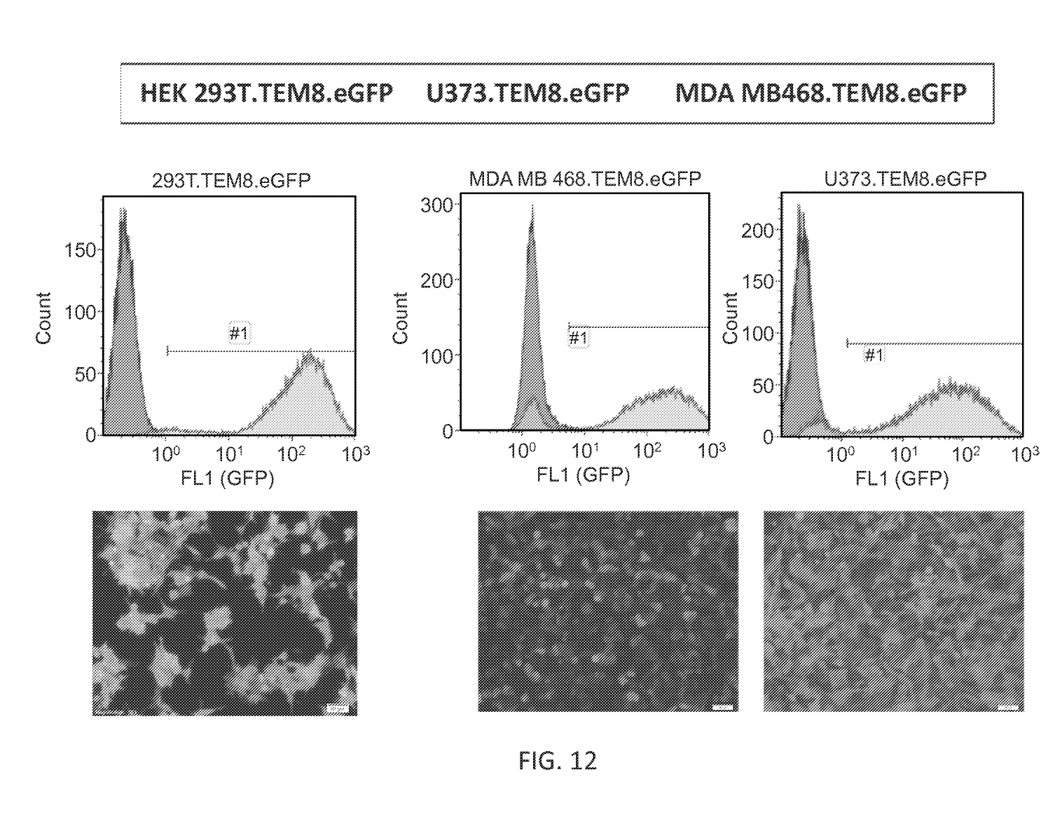

A cell line used for testing was HEK 293T, which is known in the literature to be negative for TEM8. TEM8 was force-expressed on this cell line using a lentiviral construct coding for full length TEM8 protein and an eGFP reporter gene. This same construct was used to force express TEM8 on the glioma cell line (U373) and the breast cancer cell line (MDA MB 468; a HER2 negative breast cancer cell line, for testing purposes) (FIG. 12).

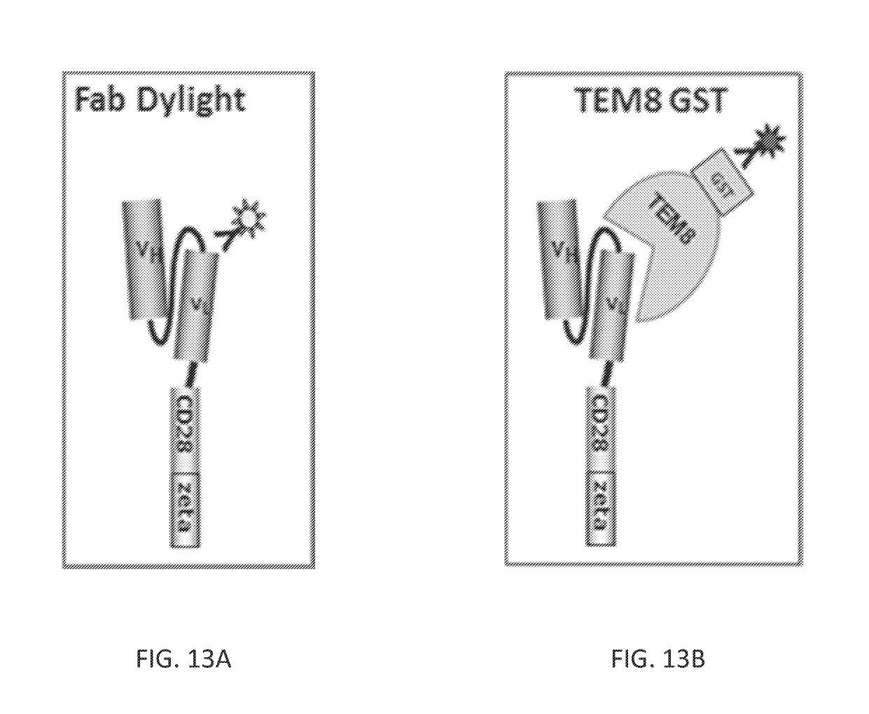

Detection of TEM8 CAR on the cell surface was performed. Two independent methods were used to detect TEM8 CAR on the cell surface. A FAB specific method; identifies a conserved region on the Vl and Vh fragments of the exodomain (FIG. 13A) or a TEM8-specific method that uses a GST tagged TEM8 protein to specifically bind TEM8 CAR. The GST fragment is tagged using a fluorophore carrying GST specific antibody (FIG. 13B).

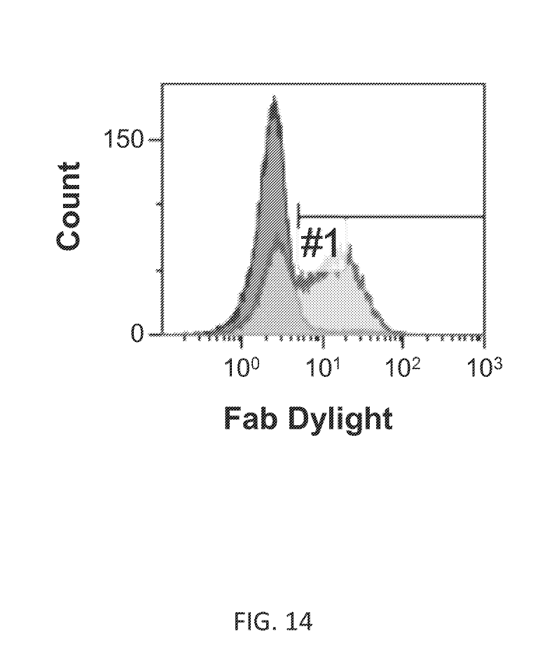

Transfection of TEM CAR into 293T cells is shown in FIG. 14. Upon synthesis of the TEM8 CAR constructs, it was subcloned into the retroviral pSFG vector. Colonies containing the construct plasmid (as indicated by selective antibiotic resistance) were then maxiprepped and transfected along with retrovirus packaging material into HEK 293T cells to generate retroviral supernatant containing TEM8 CAR DNA. Depicted in FIG. 14 is the expression of the corresponding CARs on the surface of HEK 293T Cells (non-transfected (NT) T cells serve as a control).

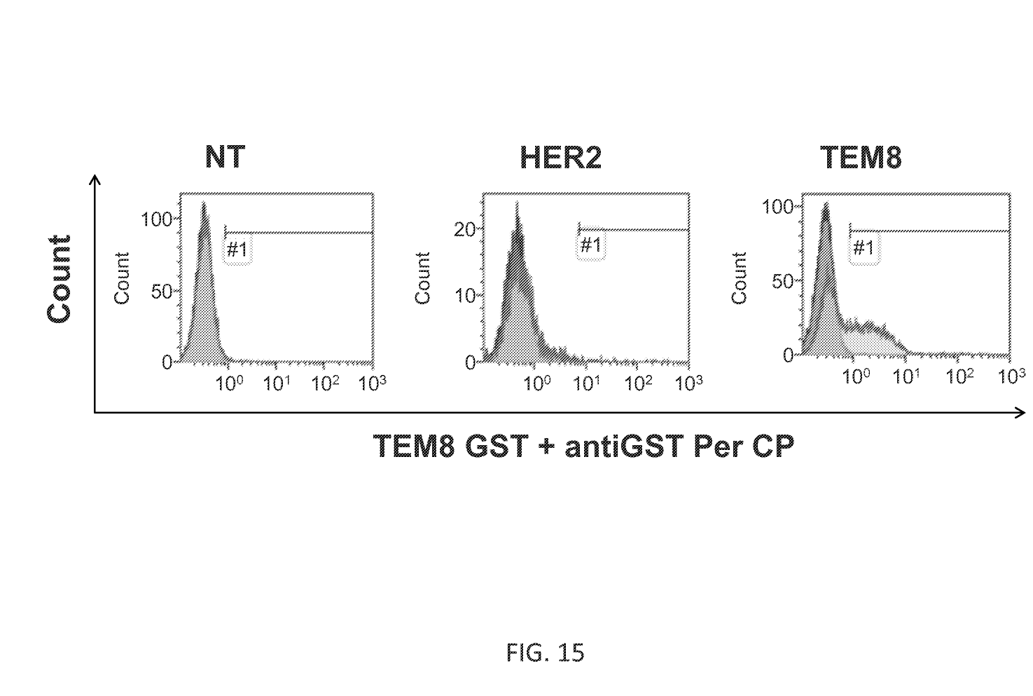

Detection of TEM8 CAR on T cells is demonstrated in FIG. 15. First, a TEM8/GST chimeric protein is incubated with the TEM8.CAR T cells, then a secondary antibody with PerCP is added to it. HER2.CAR T cells are used here as control.

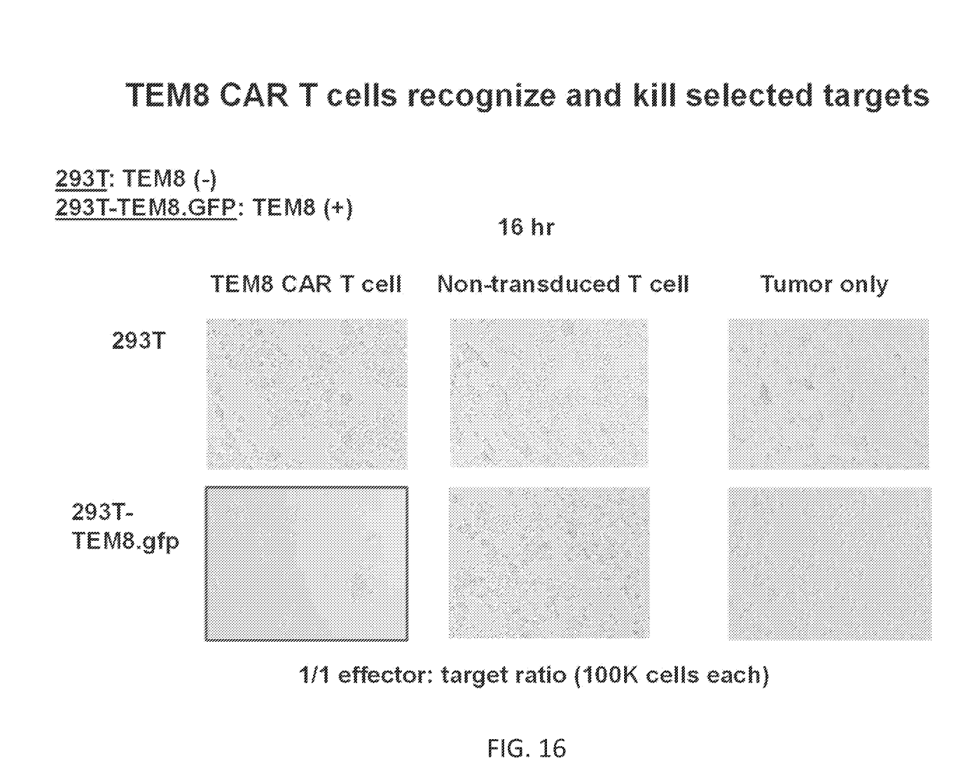

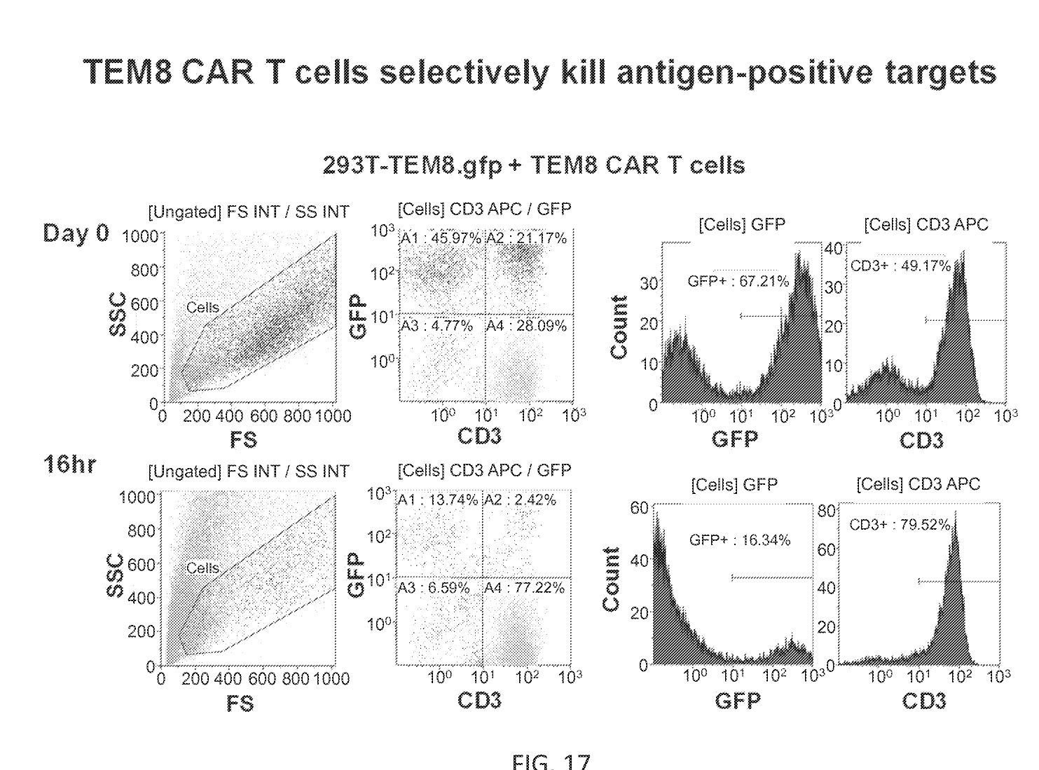

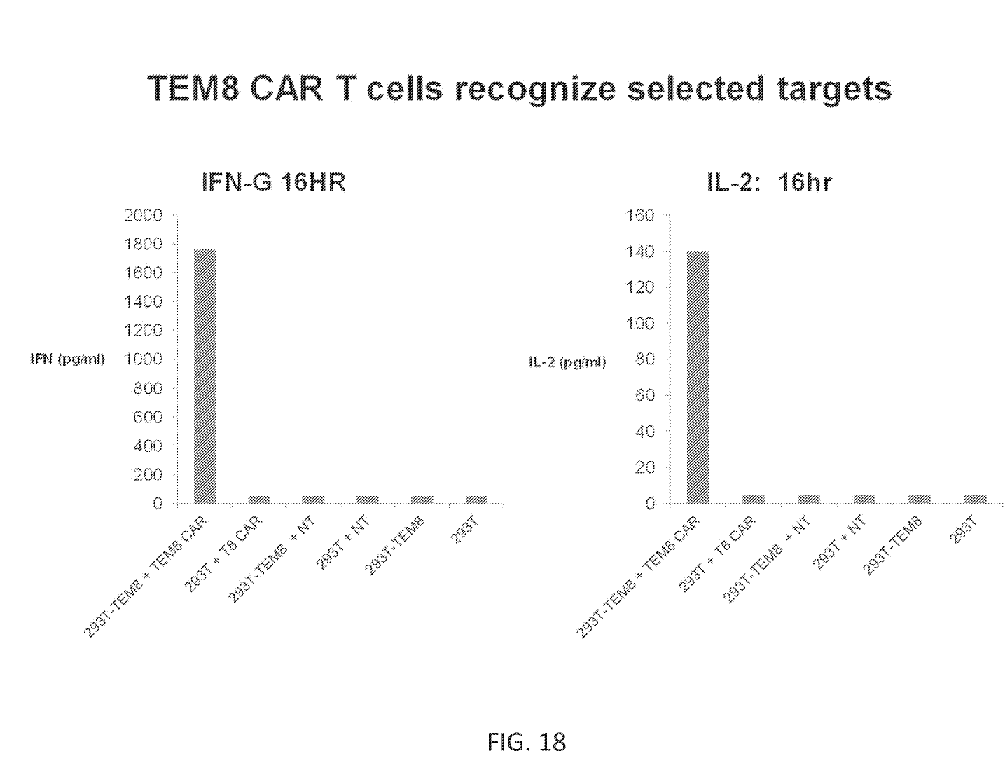

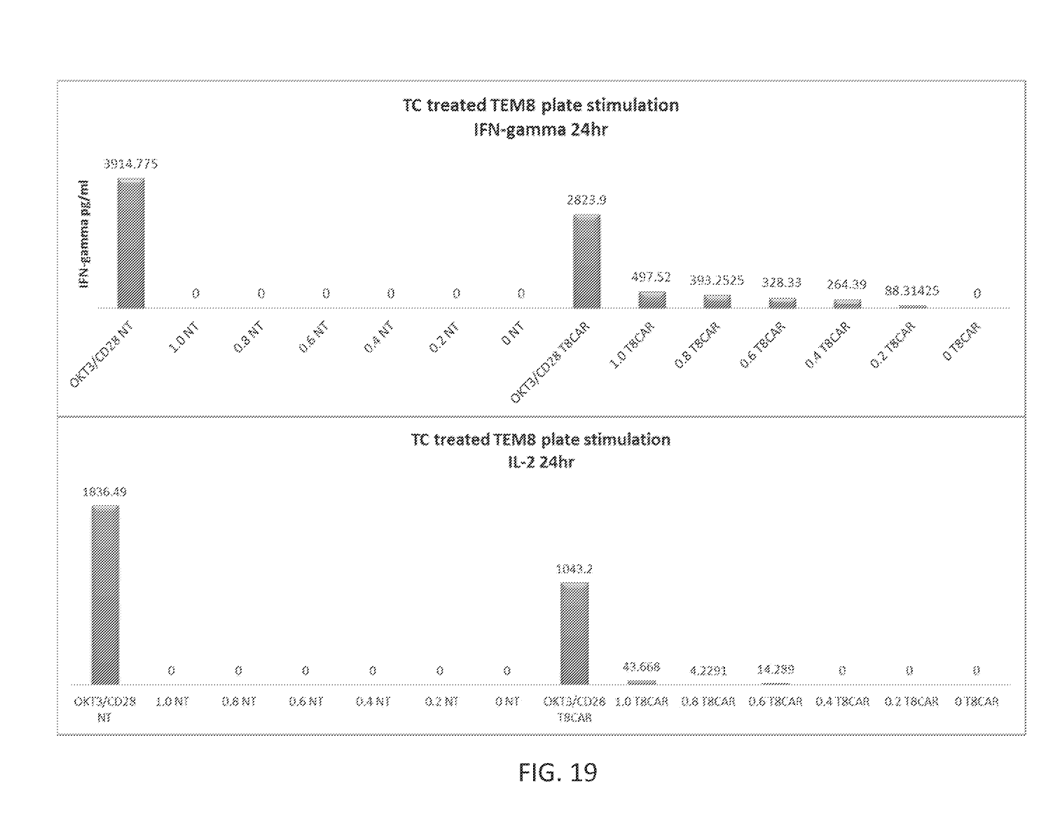

TEM8 CAR T cells recognize and kill TEM8-expressing targets. TEM8 CAR T cells were stimulated with known concentrations of recombinant TEM8 protein using a plate bound activation method. OKT3 (CD3)/CD28 antibody stimulation served as a control for stimulation. TEM8 CAR T cells were compared to non-transduced T cells (NT), which should not be stimulated by TEM8 (FIG. 16). FIG. 17 demonstrates that TEM8 CAR T cells selectively kill antigen-positive targets. TEM8 CAR T cells were stimulated with recombinant TEM8 protein using a plate bound activation method. OKT3 (CD3)/CD28 antibody stimulation served as a control for stimulation. TEM8 CAR T cells were compared to non-transduced T cells (NT), which should not be stimulated by TEM8. FIGS. 18-19 illustrate that TEM8 CAR T cells recognize selected targets.

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A polynucleotide comprising sequence that encodes a TEM1-specific chimeric antigen receptor.

2. A polynucleotide comprising sequence that encodes a TEM8-specific chimeric antigen receptor.

3. The polynucleotide of claim 1, further comprising sequence that encodes a TEM8-specific chimeric antigen receptor.

4. The polynucleotide of claim 1 or 2, wherein the chimeric antigen receptor comprises a transmembrane domain selected from the group consisting of CD3-zeta and CD28.

5. The polynucleotide of claim 1 or 2, wherein the chimeric antigen receptor comprises co-stimulatory molecule endodomains selected from the group consisting of CD28, CD27, 4-1BB, OX40 ICOS, and a combination thereof.

6. An expression vector comprising the polynucleotide of any one of claims 1 through 5.

7. The vector of claim 6, wherein the vector is a viral vector.

8. The vector of claim 7, wherein the viral vector is a retroviral vector, lentiviral vector, adenoviral vector, or adeno-associated viral vector.

9. A cell, comprising the expression vector of any one of claims 6 through 8.

10. The cell of claim 9, wherein said cell is a eukaryotic cell.

11. The cell of claim 9, wherein the cell is an immune system cell.

12. The cell of claim 9, wherein the cell is a T cell, NK cell, or NKT cell.

13. A method of treating an individual for cancer, comprising the step of providing a therapeutically effective amount of a plurality of any of cells of claims 9-12.

14. The method of claim 13, wherein the cancer is a solid tumor.

15. The method of claim 13, wherein the cancer comprises solid tumors that are about 2 mm or greater in diameter.

16. The method of claim 13, wherein the cancer is lung, bronchial, breast, prostate, intestine (including esophagus, stomach, small intestine, colon, rectal, and anal), brain and nervous system, eye (including retinoblastoma), neuroectodermal, skin, liver including bile and gallbladder, kidney, bladder, pancreatic, blood, thyroid, gynecological including cervical and ovarian, testicular, stomach, spleen, gall bladder, soft tissue (sarcoma), bone, endocrine, undifferentiated, oral cavity, head and neck, oral cavity, primary and secondary nervous system malignancies of various histologies, viral (including AIDS, EBV, HPV)-related, or undifferentiated.

17. The method of claim 13, further comprising the step of providing a therapeutically effective amount of an additional cancer therapy to the individual.

18. A kit comprising the polynucleotide of any one of claims 1 to 5, the expression vector of any one of claims 6 to 8, and/or the cells of any one of claims 9 to 12.


SEQUENCE LISTING (2 sequences)

SEQ ID NO: 1 - protein, 724 aa, Artificial Sequence
  Synthetic Peptide
MDWIWRILFLVGAATGAHSDIVMTQTPPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRGGGGSGGGGSGGGGSVQLQQSGAELVRPGVSVKISCKGSGYTFTDYAMHWVKQSHAKSLEWIGVISTYFGDATYNQKFKGKATMTVDKSSSTAYMELARLTSEDSAIYYCAREDYVPFAYWGQGTLVTVSALEDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIWVRSKRSRLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 680 aa, Artificial Sequence
  Synthetic Peptide
MDWIWRILFLVGAATGAHSEVQLVESGGGLVQPGGSLRLSCAASGFTFSSYSMNWVRQAPGKGLEWVSAISGSGGSTYYTGSVKGRFTISRDNSKNTLFLQMNSLRGENTAVYYCARYGAMNVWGQGTTVTVSSGGGGSGGGGSGGGGSQSVLTQPPSASGTPGQRVTISCSGRSPNIGSNYVYWYQFLPGTAPKLLIYRNNQRPSGVPDRFSGSKSGTSASLAISGLRSEDEADYYCAAWDDSLSGLVFGGGTQLTVLLEDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR